SEQUENCE LISTING (16 sequences)

SEQ ID NO: 1 - RNA, 57 nt, synthetic construct
  source 1..57: mol_type = other RNA
ggtgataagtggaatgccatggttgtggaaggtccagttttgaggggctattacaac

SEQ ID NO: 2 - RNA, 57 nt, synthetic construct
  source 1..57: mol_type = other RNA
gccccggccccggccccggccgttgtggaaggtccagttttgaggggctattacaac

SEQ ID NO: 3 - RNA, 57 nt, synthetic construct
  source 1..57: mol_type = other RNA
cccggccccggccccggccccgttgtggaaggtccagttttgaggggctattacaac

SEQ ID NO: 4 - RNA, 60 nt, synthetic construct
  source 1..60: mol_type = other RNA
ggccccggccccggccccggccccgttgtggaaggtccagttttgaggggctattacaac

SEQ ID NO: 5 - RNA, 51 nt, synthetic construct
  source 1..51: mol_type = other RNA
gaacccctaccaactggtcggggtttgaaacgtgataagtggaatgccatg

SEQ ID NO: 6 - RNA, 51 nt, synthetic construct
  source 1..51: mol_type = other RNA
gaacccctaccaactggtcggggtttgaaacccccggccccggccccggcc

SEQ ID NO: 7 - RNA, 53 nt, synthetic construct
  source 1..53: mol_type = other RNA
gaacccctaccaactggtcggggtttgaaacccccggccccggccccggcccc

SEQ ID NO: 8 - RNA, 55 nt, synthetic construct
  source 1..55: mol_type = other RNA
gaacccctaccaactggtcggggtttgaaacccggccccggccccggccccggcc

SEQ ID NO: 9 - RNA, 61 nt, synthetic construct
  source 1..61: mol_type = other RNA
gaacccctaccaactggtcggggtttgaaacccggccccggccccggccccggccccggcc

SEQ ID NO: 10 - RNA, 61 nt, synthetic construct
  source 1..61: mol_type = other RNA
gaacccctaccaactggtcggggtttgaaactcaccagaagcgtaccatactcacgaacag

SEQ ID NO: 11 - skipped

SEQ ID NO: 12 - protein, 20 aa, Human immunodeficiency virus
GRKKRRQRRRPPQPKKKRKV

SEQ ID NO: 13 - protein, 19 aa, Hepatitis B virus
PLSSIFSRIGDPPKKKRKV

SEQ ID NO: 14 - protein, 24 aa, synthetic construct
GALFLGWLGAAGSTMGAPKKKRKV

SEQ ID NO: 15 - protein, 27 aa, synthetic construct
GALFLGFLGAAGSTMGAWSQPKKKRKV

SEQ ID NO: 16 - protein, 21 aa, synthetic construct
KETWWETWWTEWSQPKKKRKV

(19) United States
(12) Patent Application Publication
(10) Pub. No.: US 2025/0154480 A1
(43) Pub. Date: May 15, 2025

(54) TREATMENT FOR NUCLEOTIDE REPEAT EXPANSION DISEASE

(71) Applicant: The Johns Hopkins University, Baltimore, MD (US)

(72) Inventors: Jiou Wang, Baltimore, MD (US); Honghe Liu, Baltimore, MD (US)

(21) Appl. No.: 18/838,002

(22) PCT Filed: Feb. 22, 2023
(86) PCT No.: PCT/US23/63029

§ 371 Date: Aug. 13, 2024

Related U.S. Application Data
(60) Provisional application No. 63/313,150, filed on Feb. 23, 2022.

(51) Int. Cl. C12N 9/22; A61K 38/46; A61K 48/00; A61P 25/28; C07K 14/47; C12N 15/113; C12N 15/52

(52) U.S. Cl. CPC C12N 9/22; A61K 38/465; A61K 48/005; A61P 25/28; C07K 14/47; C12N 15/113; C12N 15/52; C12N 2320/30; C12N 2510/00

(57) ABSTRACT

Provided herein is technology relating to nucleotide repeat elements present in the genomes of eukaryotes and particularly, but not exclusively, to technologies for reducing the levels of disease-causing products expressed from expansions of nucleotide repeats.

### TREATMENT FOR NUCLEOTIDE REPEAT EXPANSION DISEASE STATEMENT OF RELATED APPLICATIONS

This application claims priority to U.S. Provisional Patent Application No. 63/313,150, filed Feb. 23, 2022, the entire contents of which are incorporated herein by reference for all purposes.

### STATEMENT REGARDING FEDERALLY SPONSORED RESEARCH OR DEVELOPMENT

This invention was made with government support under grant NS089616 awarded by the National Institute of Health. The government has certain rights in the invention.

### SEQUENCE LISTING

The text of the computer readable sequence listing filed herewith, titled “JHU-40604-601_SQL”, created Feb. 22, 2023, having a file size of 14,814 bytes, is hereby incorporated by reference in its entirety.

### FIELD

Provided herein is technology relating to nucleotide repeat elements present in the genomes of eukaryotes and particularly, but not exclusively, to technologies for reducing the levels of disease-causing products expressed from expansions of nucleotide repeats.

### BACKGROUND

Nucleotide repeat elements (e.g., microsatellites, short tandem repeats, etc.) are common in the genomes of eukaryotes including humans (see, e.g., Richard (2008) “Comparative genomics and molecular dynamics of DNA repeats in eukaryotes” Microbiology and Molecular Biology Reviews 72: 686-727, incorporated herein by reference). Expansions of nucleotide repeat elements (e.g., short nucleotide repeats) have been linked to nearly 50 different types of genetic disorders, primarily neurological and neuromuscular disorders (see, e.g., Pearson (2005) “Repeat instability: mechanisms of dynamic mutations” Nat Rev Genet 6: 729-742; La Spada (2010) “Repeat expansion disease: progress and puzzles in disease pathogenesis” Nat Rev Genet 11: 247-258; and Khristich (2020) “On the wrong DNA track: Molecular mechanisms of repeat-mediated genome instability” J Biol Chem 295: 4134-4170, each of which is incorporated herein by reference). Toxicities produced by repeat-containing RNAs and/or their protein products are important causes of pathogenetic processes (see, e.g., Pearson, La Spada, and Khristich, supra) and associated disease states. Minimizing and/or eliminating products expressed from nucleotide repeat expansions would provide a method for treatment of diseases caused by these expression products. However, no effective treatments exist for these diseases in part due to the fact that the nucleotide repeats are difficult to target by conventional technologies.

### SUMMARY

Accordingly, provided herein is a technology related to minimizing and/or eliminating the expression products of nucleotide repeat expansions (e.g., repeat-containing RNA and/or proteins translated from repeat-containing RNA) as a treatment of disease caused by the expression products of nucleotide repeat expansions (e.g., repeat-containing RNA and/or proteins translated from repeat-containing RNA).

Accordingly, provided herein is a technology related to a ribonucleoprotein (RNP) comprising a Cas13 protein and a gRNA, wherein said gRNA comprises a spacer sequence comprising one or more CCGGCC sequences. In some embodiments, the Cas13 protein is Cas13d. In some embodiments, the spacer sequence is 24 nt long. In some embodiments, the spacer sequence is 30 nt long. In some embodiments, the gRNA sequence comprises a G or A at the +1 position. In some embodiments, the gRNA comprises a sequence provided by SEQ ID NO: 8. In some embodiments, the gRNA comprises a sequence provided by SEQ ID NO: 9.

In some embodiments, provided herein is a technology related to a ribonucleoprotein (RNP) comprising a Cas13 protein and a gRNA, wherein said gRNA comprises a spacer sequence comprising one or more CCCCGG sequences. In some embodiments, the Cas13 protein is Cas13d. In some embodiments, the spacer sequence is 24 nt long. In some embodiments, the spacer sequence is 30 nt long. In some embodiments, the gRNA sequence comprises a G or A at the +1 position. In some embodiments, the gRNA comprises a sequence provided by SEQ ID NO 8. In some embodiments, the gRNA comprises a sequence provided by SEQ ID NO: 9.

In some embodiments, the technology relates to a gRNA comprising a spacer sequence comprising one or more CCCCGG and/or CCGGCC sequences. In some embodiments, the spacer sequence is 24 nt long. In some embodiments, the spacer sequence is 30 nt long. In some embodiments, the gRNA sequence comprises a G or A at the +1 position. In some embodiments, the gRNA comprises a sequence provided by SEQ ID NO: 8. In some embodiments, the gRNA comprises a sequence provided by SEQ ID NO: 9.

Further, in some embodiments, the technology provides a nucleic acid comprising a first nucleotide sequence encoding a Cas13 protein and a second nucleotide sequence encoding a gRNA, wherein said gRNA comprises a spacer sequence comprising one or more CCGGCC sequences. In some embodiments, the Cas13 protein is Cas13d. In some embodiments, the spacer sequence is 24 nt long. In some embodiments, the spacer sequence is 30 nt long. In some embodiments, the gRNA sequence comprises a G or A at the +1 position. In some embodiments, the gRNA comprises a sequence provided by SEQ ID NO: 8. In some embodiments, the gRNA comprises a sequence provided by SEQ ID NO: 9.

In some embodiments, the technology provides a vector comprising a nucleic acid described herein (e.g., a nucleic acid comprising a first nucleotide sequence encoding a Cas13 protein and a second nucleotide sequence encoding a gRNA, wherein said gRNA comprises a spacer sequence comprising one or more CCGGCC sequences). In some embodiments, the technology provides a cell comprising a nucleic acid as described herein (e.g., a nucleic acid comprising a first nucleotide sequence encoding a Cas13 protein and a second nucleotide sequence encoding a gRNA, wherein said gRNA comprises a spacer sequence comprising one or more CCGGCC sequences). In some embodiments, a cell comprises a vector comprising a nucleic acid described herein (e.g., a nucleic acid comprising a first nucleotide sequence encoding a Cas13 protein and a second nucleotide sequence encoding a gRNA, wherein said gRNA comprises a spacer sequence comprising one or more CCGGCC sequences). In some embodiments, the cell is a human cell comprising a hexanucleotide repeat in chromosome 9. In some embodiments, the cell is a human cell comprising a hexanucleotide repeat at a C9orf72 locus. In some embodiments, the cell is a human cell comprising one or more GGGGCC repeats (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, or more GGGGCC repeats) at a C9orf72 locus. In some embodiments, a patient having a neurological disease comprises the cell. In some embodiments, a patient having amyotrophic lateral sclerosis comprises the cell. In some embodiments, a patient having frontotemporal dementia comprises the cell. In some embodiments, a patient having Alzheimer's disease comprises the cell. In some embodiments, a patient having Huntington's disease, multiple system atrophy, depressive pseudo dementia, or bipolar disorder comprises the cell.

In some embodiments, the technology provides a cell comprising an RNP as described herein (e.g., a ribonucleoprotein (RNP) comprising a Cas13 protein and a gRNA, wherein said gRNA comprises a spacer sequence comprising one or more CCGGCC sequences). In some embodiments, the cell is a human cell comprising a hexanucleotide repeat in chromosome 9. In some embodiments, the cell is a human cell comprising a hexanucleotide repeat at a C9orf72 locus. In some embodiments, the cell is a human cell comprising one or more GGGGCC repeats (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, or more GGGGCC repeats) at a C9orf72 locus. In some embodiments, a patient having a neurological disease comprises the cell. In some embodiments, a patient having amyotrophic lateral sclerosis comprises the cell. In some embodiments, a patient having frontotemporal dementia comprises the cell. In some embodiments, a patient having Alzheimer's disease comprises the cell. In some embodiments, a patient having Huntington's disease, multiple system atrophy, depressive pseudo dementia, or bipolar disorder comprises the cell.

In some embodiments, the technology provides a system comprising a Cas13 protein and a gRNA, wherein said gRNA comprises a spacer sequence comprising one or more CCGGCC sequences. In some embodiments, the technology provides a system comprising a nucleic acid encoding a Cas13 protein and a gRNA, wherein said gRNA comprises a spacer sequence comprising one or more CCGGCC sequences. In some embodiments, the technology provides a system comprising a Cas13 protein and a nucleic acid encoding a gRNA, wherein said gRNA comprises a spacer sequence comprising one or more CCGGCC sequences. In some embodiments, the technology provides a system comprising a nucleic acid encoding a Cas13 protein and a nucleic acid encoding a gRNA, wherein said gRNA comprises a spacer sequence comprising one or more CCGGCC sequences. In some embodiments, the Cas13 protein is Cas13d. In some embodiments, the spacer sequence is 24 nt long. In some embodiments, the spacer sequence is 30 nt long. In some embodiments, the gRNA sequence comprises a G or A at the +1 position. In some embodiments, the gRNA comprises a sequence provided by SEQ ID NO: 8. In some embodiments, the gRNA comprises a sequence provided by SEQ ID NO: 9.

In some embodiments, the technology provides a method of treating a subject having a neurological disease, said method comprising administering a ribonucleoprotein (RNP) to said subject, wherein said RNP comprises a Cas13 protein and a gRNA and wherein said gRNA comprises a spacer sequence comprising one or more CCGGCC sequences. In some embodiments, the Cas13 protein is Cas13d. In some embodiments, the spacer sequence is 24 nt long. In some embodiments, the spacer sequence is 30 nt long. In some embodiments, the gRNA sequence comprises a G or A at the +1 position. In some embodiments, the gRNA comprises a sequence provided by SEQ ID NO: 8. In some embodiments, the gRNA comprises a sequence provided by SEQ ID NO: 9. In some embodiments, the neurological disease is amyotrophic lateral sclerosis. In some embodiments, the neurological disease is frontotemporal dementia. In some embodiments, the neurological disease is Alzheimer's disease. In some embodiments, the neurological disease is Huntington's disease, multiple system atrophy, depressive pseudo dementia, or bipolar disorder. In some embodiments, the subject comprises a cell comprising a hexanucleotide repeat in chromosome 9. In some embodiments, the subject 25 comprises a cell comprising a hexanucleotide repeat at a C9orf72 locus. In some embodiments, the subject comprises a cell comprising one or more GGGGCC repeats at a C9orf72 locus (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, or more GGGGCC repeats).

In some embodiments, the technology provides a method of treating a subject having a neurological disease, said method comprising administering a nucleic acid to said subject, wherein said nucleic acid comprises a first nucleotide sequence encoding a Cas13 protein and a second nucleotide sequence encoding a gRNA and wherein said gRNA comprises a spacer sequence comprising one or more CCGGCC sequences. In some embodiments, the Cas13 protein is Cas13d. In some embodiments, the spacer sequence is 24 nt long. In some embodiments, the spacer sequence is 30 nt long. In some embodiments, the gRNA sequence comprises a G or A at the +1 position. In some embodiments, the gRNA comprises a sequence provided by SEQ ID NO: 8. In some embodiments, the gRNA comprises a sequence provided by SEQ ID NO: 9. In some embodiments, the neurological disease is amyotrophic lateral sclerosis. In some embodiments, the neurological disease is frontotemporal dementia. In some embodiments, the neurological disease is Alzheimer's disease. In some embodiments, the neurological disease is Huntington's disease, multiple system atrophy, depressive pseudo dementia, or bipolar disorder. In some embodiments, the subject comprises a cell comprising a hexanucleotide repeat in chromosome 9. In some embodiments, the subject comprises a cell comprising a hexanucleotide repeat at a C9orf72 locus. In some embodiments, the subject comprises a cell comprising one or more GGGGCC repeats at a C9orf72 locus (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, or more GGGGCC repeats).

Additional embodiments will be apparent to persons skilled in the relevant art based on the teachings contained herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

These and other features, aspects, and advantages of the present technology will become better understood with regard to the following drawings.

FIG. 1A, FIG. 1B, FIG. 1C, and FIG. 1D show the CRISPR-Cas13d one-vector system scheme and knock-down efficiency test with an EGFP reporter plasmid. FIG. 1A is a schematic showing a one vector CRISPR-Cas13d construct. FIG. 1B is a schematic of a reporter plasmid. FIG. 1C shows the results of a knock-down test in HEK 293 cells by co-transfection of CRISPR-Cas13d vector and reporter plasmid. FIG. 1D is a bar plot quantifying the results shown in FIG. 1C. Data are provided as means±SD of three independent experiments. *P<0.05, **P<0.01.

FIG. 2A and FIG. 2B show that Cas13d/gRNA specifically decreased translation of GGGGCC repeats in a luciferase reporter construct. FIG. 2A is a schematic of the inducible luciferase-based C9orf72 RAN translation reporter construct (top) and control (bottom, “No insert”). FIG. 2B shows the results from experiments using reporter cells stably expressing Cas13d and a S24 gRNA or a S30 gRNA. The data indicated a lower Nano and Firefly luciferase signal in (GGGGCC)70 containing cells but not in control cells. Data are given as means±SD of four replicates from two independent experiments. **P<0.01, ****P<0.0001.

FIG. 3A, FIG. 3B, FIG. 3C, and FIG. 3D show that Cas13d/gRNA significantly decreases GP level in human cells derived from C9orf72 linked patients. FIG. 3A to FIG. 3D show the results from experiments in which human iPSC cells stably expressed Cas13d and gRNA by lentivirus transduction. The GP levels were quantified by ELISA. Data are provided as means±SD of four or two replicates from two independent experiments. *P<0.05, **P<0.01, ***P<0.001, ****P<0.0001.

FIG. 4A, FIG. 4B, FIG. 4C, FIG. 4D, and FIG. 4E show that Cas13d/gRNA significantly decreases GP level in human motor neuron cells derived from C9orf72 linked patients. FIG. 4A to FIG. 4E show the results from experiments in which motor neuron cells differentiated from five human iPSC lines derived from patients stably expressed Cas13d and gRNA by lentivirus transduction. The GP levels were quantified by ELISA. Data are provided as means±SD of four replicates from two independent experiments. **P<0.01, ***P<0.001, ****P<0.0001.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

Provided herein is technology relating to nucleotide repeat elements present in the genomes of eukaryotes and particularly, but not exclusively, to technologies for reducing the levels of disease-causing products expressed from expansions of nucleotide repeats.

In this detailed description of the various embodiments, for purposes of explanation, numerous specific details are set forth to provide a thorough understanding of the embodiments disclosed. One skilled in the art will appreciate, however, that these various embodiments may be practiced with or without these specific details. In other instances, structures and devices are shown in block diagram form. Furthermore, one skilled in the art can readily appreciate that the specific sequences in which methods are presented and performed are illustrative and it is contemplated that the sequences can be varied and still remain within the spirit and scope of the various embodiments disclosed herein.

All literature and similar materials cited in this application, including but not limited to, patents, patent applications, articles, books, treatises, and internet web pages are expressly incorporated by reference in their entirety for any purpose. Unless defined otherwise, all technical and scientific terms used herein have the same meaning as is commonly understood by one of ordinary skill in the art to which the various embodiments described herein belongs. When definitions of terms in incorporated references appear to differ from the definitions provided in the present teachings, the definition provided in the present teachings shall control. The section headings used herein are for organizational purposes only and are not to be construed as limiting the described subject matter in any way.

### Definitions

To facilitate an understanding of the present technology, a number of terms and phrases are defined below. Additional definitions are set forth throughout the detailed description.

Throughout the specification and claims, the following terms take the meanings explicitly associated herein, unless the context clearly dictates otherwise. The phrase “in some embodiments” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

In addition, as used herein, the term “or” is an inclusive “or” operator and is equivalent to the term “and/or” unless the context clearly dictates otherwise. The term “based on” is not exclusive and allows for being based on additional factors not described, unless the context clearly dictates otherwise. In addition, throughout the specification, the meaning of “a”, “an”, and “the” include plural references. The meaning of “in” includes “in” and “on.”

As used herein, the terms “about”, “approximately”, “substantially”, and “significantly” are understood by persons of ordinary skill in the art and will vary to some extent on the context in which they are used. If there are uses of these terms that are not clear to persons of ordinary skill in the art given the context in which they are used, “about” and “approximately” mean plus or minus less than or equal to 10% of the particular term and “substantially” and “significantly” mean plus or minus greater than 10% of the particular term.

As used herein, disclosure of ranges includes disclosure of all values and further divided ranges within the entire range, including endpoints and sub-ranges given for the ranges. As used herein, the disclosure of numeric ranges includes the endpoints and each intervening number therebetween with the same degree of precision. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9, and 7.0 are explicitly contemplated.

As used herein, the suffix “-free” refers to an embodiment of the technology that omits the feature of the base root of the word to which “-free” is appended. That is, the term “X-free” as used herein means “without X”, where X is a feature of the technology omitted in the “X-free” technology. For example, a “calcium-free” composition does not comprise calcium, a “mixing-free” method does not comprise a mixing step, etc.

Although the terms “first”, “second”, “third”, etc. may be used herein to describe various steps, elements, compositions, components, regions, layers, and/or sections, these steps, elements, compositions, components, regions, layers, and/or sections should not be limited by these terms, unless otherwise indicated. These terms are used to distinguish one step, element, composition, component, region, layer, and/or section from another step, element, composition, component, region, layer, and/or section. Terms such as “first”, “second”, and other numerical terms when used herein do not imply a sequence or order unless clearly indicated by the context. Thus, a first step, element, composition, component, region, layer, or section discussed herein could be termed a second step, element, composition, component, region, layer, or section without departing from technology.

As used herein, the word “presence” or “absence” (or, alternatively, “present” or “absent”) is used in a relative sense to describe the amount or level of a particular entity (e.g., an analyte). For example, when an analyte is said to be “present” in a test sample, it means the level or amount of this analyte is above a pre-determined threshold; conversely, when an analyte is said to be “absent” in a test sample, it means the level or amount of this analyte is below a pre-determined threshold. The pre-determined threshold may be the threshold for detectability associated with the particular test used to detect the analyte or any other threshold. When an analyte is “detected” in a sample it is “present” in the sample; when an analyte is “not detected” it is “absent” from the sample. Further, a sample in which an analyte is “detected” or in which the analyte is “present” is a sample that is “positive” for the analyte. A sample in which an analyte is “not detected” or in which the analyte is “absent” is a sample that is “negative” for the analyte.

As used herein, an “increase” or a “decrease” refers to a detectable (e.g., measured) positive or negative change, respectively, in the value of a variable relative to a previously measured value of the variable, relative to a pre-established value, and/or relative to a value of a standard control. An increase is a positive change preferably at least 10%, more preferably 50%, still more preferably 2-fold, even more preferably at least 5-fold, and most preferably at least 10-fold relative to the previously measured value of the variable, the pre-established value, and/or the value of a standard control. Similarly, a decrease is a negative change preferably at least 10%, more preferably 50%, still more preferably at least 80%, and most preferably at least 90% of the previously measured value of the variable, the pre-established value, and/or the value of a standard control. Other terms indicating quantitative changes or differences, such as “more” or “less,” are used herein in the same fashion as described above.

As used herein, a “system” refers to a plurality of components operating together for a common purpose. In some embodiments, each component of the system interacts with one or more other components and/or is related to one or more other components.

As used herein, the term “administration” refers to providing or giving a subject an agent, such as a Cas13 protein (or Cas13 coding sequence) or guide molecule (or coding sequence) disclosed herein, by any effective route. Exemplary routes of administration include, but are not limited to, injection (such as subcutaneous, intramuscular, intradermal, intraperitoneal, intratumoral, and intravenous), transdermal, intranasal, and inhalation routes.

As used herein, the term “Cas 13” refers to an RNA-guided RNA endonuclease enzyme that can cut or bind RNA. Cas13 proteins comprise one or two HEPN domains. Native HEPN domains include the sequence RXXXXH (SEQ ID NO: 11). In addition, Cas13 proteins specifically recognize direct repeat sequences of gRNA having a particular secondary structure. In one example, Cas13 proteins recognize and/or bind a direct repeat (DR) sequence comprising (1) a loop of approximately 4 to 8 nt; (2) a stem of 4 to 12 nt formed of complementary nucleotides, which can include a small (e.g., 1 or 2 bp) bulge due to a nt mismatch in the stem; and (3) a bulge or overhang formed of unpaired nucleotides, which can be approximately 10 to 14 nt (e.g., 5 to 7 on each side). In one example, the full length (non-truncated) Cas13 protein is between 870-1080 amino acids long. In one example, the corresponding DR sequence of a Cas13d protein is located at the 5′ end of the spacer sequence in the molecule that includes the Cas13 gRNA. In one example, the DR sequence in the Cas13 gRNA is truncated at the 5′ end relative to the DR sequence in the unprocessed Cas13 guide array transcript. In one example, the DR sequence in the Cas13 gRNA is truncated by 5-7 nt at the 5′ end by the Cas13d protein. In one example, the Cas13 protein can cut a target RNA flanked at the 3′ end of the spacer-target duplex by any of a A, U, G, or C ribonucleotide and flanked at the 5′ end by any of a A, U, G, or C ribonucleotide. In some embodiments, the Cas13 protein is Cas13d, Cas13Rx, another Cas13 protein described herein or known in the art, or a protein having an activity similar to a Cas13 protein such as, e.g., Cas13d, Cas13Rx, or another Cas13 protein described herein or known in the art.

As used herein, the term “Repeat Associated Non-AUG translation” or “RAN translation” refers to a mode of mRNA translation that can occur in eukaryotic cells comprising repeat sequences.

As used herein, a “nucleic acid” or a “nucleic acid sequence” refers to a polymer or oligomer of pyrimidine and/or purine bases, preferably cytosine, thymine, and uracil, and adenine and guanine, respectively (See Albert L. Lehninger, Principles of Biochemistry, at 793-800 (Worth Pub. 1982), incorporated herein by reference). The present technology contemplates any deoxyribonucleotide, ribonucleotide, or peptide nucleic acid component, and any chemical variants thereof, such as methylated, hydroxymethylated, or glycosylated forms of these bases, and the like. The polymers or oligomers may be heterogenous or homogenous in composition and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. In some embodiments, a nucleic acid or nucleic acid sequence comprises other kinds of nucleic acid structures such as, for instance, a DNA/RNA helix, peptide nucleic acid (PNA), morpholino nucleic acid (see, e.g., Braasch and Corey, Biochemistry, 2002, 41(14), 4503-4510, incorporated herein by reference) and U.S. Pat. No. 5,034,506, incorporated herein by reference), locked nucleic acid (LNA: see Wahlestedt et al., Proc. Natl. Acad. Sci. U.S.A., 2000, 97, 5633-5638, incorporated herein by reference), cyclohexenyl nucleic acids (see Wang, J. Am. Chem. Soc., 2000, 122, 8595-8602, incorporated herein by reference), and/or a ribozyme. Hence, the term “nucleic acid” or “nucleic acid sequence” may also encompass a chain comprising non-natural nucleotides, modified nucleotides, and/or non- nucleotide building blocks that can exhibit the same function as natural nucleotides (e.g., “nucleotide analogs”); further, the term “nucleic acid sequence” as used herein refers to an oligonucleotide, nucleotide or polynucleotide, and fragments or portions thereof, and to DNA or RNA of genomic or synthetic origin, which may be single or double-stranded, and represent the sense or antisense strand.

Furthermore, the terms “nucleic acid”, “polynucleotide”, “nucleotide sequence”, and “oligonucleotide” are used interchangeably. They refer to a polymeric form of nucleotides of any length, either deoxyribonucleotides or ribonucleotides, or analogs thereof. Polynucleotides may have any three-dimensional structure and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. The term also encompasses nucleic-acid-like structures with synthetic backbones, see, e.g., Eckstein, 1991; Baserga et al., 1992; Milligan, 1993; WO 97/03211; WO 96/39154; Mata, 1997; Strauss-Soukup, 1997; and Samstag, 1996, each of which is incorporated herein by reference. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

The term “nucleotide analog” as used herein refers to modified or non-naturally occurring nucleotides including but not limited to analogs that have altered stacking interactions such as 7-deaza purines (i.e., 7-deaza-dATP and 7-deaza-dGTP); base analogs with alternative hydrogen bonding configurations (e.g., such as Iso-C and Iso-G and other non-standard base pairs described in U.S. Pat. No. 6,001,983 to S. Benner, herein incorporated by reference); non-hydrogen bonding analogs (e.g., non-polar, aromatic nucleoside analogs such as 2,4-difluorotoluene, described by B. A. Schweitzer and E. T. Kool, J. Org. Chem., 1994, 59, 7238-7242, B. A. Schweitzer and E. T. Kool, J. Am. Chem. Soc., 1995, 117, 1863-1872; each of which is herein incorporated by reference); “universal” bases such as 5-nitroindole and 3-nitropyrrole; and universal purines and pyrimidines (such as “K” and “P” nucleotides, respectively; P. Kong, et al., Nucleic Acids Res., 1989, 17, 10373-10383, P. Kong et al., Nucleic Acids Res., 1992, 20, 5149-5152, each of which is incorporated herein by reference). Nucleotide analogs include nucleotides having modification on the sugar moiety, such as dideoxy nucleotides and 2′-O-methyl nucleotides. Nucleotide analogs include modified forms of deoxyribonucleotides as well as ribonucleotides.

“Peptide nucleic acid” means a DNA mimic that incorporates a peptide-like polyamide backbone.

As used herein, the term “% sequence identity” refers to the percentage of nucleotides or nucleotide analogs in a nucleic acid sequence that is identical with the corresponding nucleotides in a reference sequence after aligning the two sequences and introducing gaps, if necessary, to achieve the maximum percent identity. Hence, in case a nucleic acid according to the technology is longer than a reference sequence, additional nucleotides in the nucleic acid, that do not align with the reference sequence, are not taken into account for determining sequence identity. Methods and computer programs for alignment are well known in the art, including BLAST, Align 2, and FASTA.

The term “homology” and “homologous” refers to a degree of identity. There may be partial homology or complete homology. A partially homologous sequence is one that is less than 100% identical to another sequence.

The term “sequence variation” as used herein refers to a difference or multiple differences in nucleic acid sequence between two nucleic acids. For example, a wild-type structural gene and a mutant form of this wild-type structural gene may vary in sequence by the presence of one or more single base substitutions or by deletions and/or insertions of one or more nucleotides. These two forms of the structural gene are said to vary in sequence from one another. A second mutant form of the structural gene may exist. This second mutant form is said to vary in sequence from both the wild-type gene and the first mutant form of the gene.

As used herein, the terms “complementary”, “hybridizable”, or “complementarity” are used in reference to polynucleotides (e.g., a sequence of nucleotides such as an oligonucleotide or a target nucleic acid) related by the base-pairing rules. For example, for the sequence “5′-A-G-T-3′” is complementary to the sequence “3′-T-C-A-5′.” Complementarity may be “partial,” in which only some of the nucleic acid bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids. Either term may also be used in reference to individual nucleotides, especially within the context of polynucleotides. For example, a particular nucleotide within an oligonucleotide may be noted for its complementarity, or lack thereof, to a nucleotide within another nucleic acid strand, in contrast or comparison to the complementarity between the rest of the oligonucleotide and the nucleic acid strand.

In some contexts, the term “complementarity” and related terms (e.g., “complementary”, “complement”) refers to the nucleotides of a nucleic acid sequence that can bind to another nucleic acid sequence through hydrogen bonds, e.g., nucleotides that are capable of base pairing, e.g., by Watson-Crick base pairing or other base pairing. Nucleotides that can form base pairs, e.g., nucleotides that are complementary to one another, are the pairs: cytosine and guanine, thymine and adenine, adenine and uracil, and guanine and uracil. The percentage complementarity need not be calculated over the entire length of a nucleic acid sequence. The percentage of complementarity may be limited to a specific region of which the nucleic acid sequences that are base-paired, e.g., starting from a first base-paired nucleotide and ending at a last base-paired nucleotide. The complement of a nucleic acid sequence as used herein refers to an oligonucleotide which, when aligned with the nucleic acid sequence such that the 5′ end of one sequence is paired with the 3′ end of the other, is in “antiparallel association.” Certain bases not commonly found in natural nucleic acids may be included in the nucleic acids of the present invention and include, for example, inosine and 7-deazaguanine. Complementarity need not be perfect; stable duplexes may contain mismatched base pairs or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs.

It is understood in the art that the sequence of a polynucleotide need not be 100% complementary to that of its target nucleic acid to be hybridizable or specifically hybridizable. Moreover, a polynucleotide may hybridize over one or more segments such that intervening or adjacent segments are not involved in the hybridization event (e.g., a loop structure or hairpin structure). A polynucleotide can comprise at least 70%, at least 80%, at least 90%, at least 95%, at least 99%, or 100% sequence complementarity to a target region within the target nucleic acid sequence to which they are targeted. For example, a nucleic acid in which 18 of 20 nucleotides of the nucleic acid are complementary to a target region, and would therefore specifically hybridize, would represent 90 percent complementarity. In this example, the remaining non-complementary nucleotides may be clustered or interspersed with complementary nucleotides and need not be contiguous to each other or to complementary nucleotides. Percent complementarity between particular segments of nucleic acid sequences within nucleic acids can be determined routinely using BLAST programs (basic local alignment search tools) and PowerBLAST programs known in the art (Altschul et al., J. Mol. Biol., 1990, 215, 403-410; Zhang and Madden, Genome Res., 1997, 7, 649-656, each of which is incorporated herein by reference) or by using the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489, incorporated herein by reference).

Thus, in some embodiments, “complementary” refers to a first nucleobase sequence that is at least 60%, 65%, 70%, 75%, 80%, 85%, 90%, 95%, 97%, 98%, or 99% identical to the complement of a second nucleobase sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, or more nucleobases, or that the two sequences hybridize under stringent hybridization conditions. “Fully complementary” means each nucleobase of a first nucleic acid is capable of pairing with each nucleobase at a corresponding position in a second nucleic acid. For example, in certain embodiments, an oligonucleotide wherein each nucleobase has complementarity to a nucleic acid has a nucleobase sequence that is identical to the complement of the nucleic acid over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, or more nucleobases.

As used herein, the term “mismatch” refers to a nucleobase of a first nucleic acid that is not capable of pairing with a nucleobase at a corresponding position of a second nucleic acid.

As used herein, the term “hybridization” is used in reference to the pairing of complementary nucleic acids or complementary portions of one or more nucleic acid/s. Hybridization and the strength of hybridization (i.e., the strength of the association between the nucleic acids) is influenced by such factors as the degree of complementary between the nucleic acids, stringency of the conditions involved, and the Tm of the formed hybrid. “Hybridization” methods involve the annealing of one nucleic acid to another, complementary nucleic acid, e.g., a nucleic acid having a complementary nucleotide sequence. The ability of two polymers of nucleic acid containing complementary sequences to find each other and “anneal” or “hybridize” through base pairing interaction is a well-recognized phenomenon. The initial observations of the “hybridization” process by Marmur and Lane, Proc. Natl. Acad. Sci. USA 46:453 (1960) and Doty et al., Proc. Natl. Acad. Sci. USA 46:461 (1960), each of which is incorporated herein by reference, have been followed by the refinement of this process into an essential tool of modern biology. For example, hybridization and washing conditions are now well known and exemplified in Sambrook, J., Fritsch, E. F. and Maniatis, T. Molecular Cloning: A Laboratory Manual. Second Edition, Cold Spring Harbor Laboratory Press, Cold Spring Harbor (1989), particularly Chapter 11 and Table 11.1 therein; and Sambrook, J. and Russell, W., Molecular Cloning: A Laboratory Manual, Third Edition, Cold Spring Harbor Laboratory Press, Cold Spring Harbor (2001), each of which is incorporated herein by reference. The conditions of temperature and ionic strength determine the “stringency” of the hybridization.

As used herein, a “double-stranded nucleic acid” may be a portion of a nucleic acid, a region of a longer nucleic acid, or an entire nucleic acid. A “double-stranded nucleic acid” may be, e.g., without limitation, a double-stranded DNA, a double-stranded RNA, a double-stranded DNA/RNA hybrid, etc. A single-stranded nucleic acid having secondary structure (e.g., base-paired secondary structure) and/or higher order structure (e.g., a stem-loop structure) comprises a “double-stranded nucleic acid”. For example, triplex structures are considered to be “double-stranded”. In some embodiments, any base-paired nucleic acid is a “double-stranded nucleic acid”.

As used herein, the term “genomic locus” or “locus” (plural “loci”) is the specific location of a gene or DNA sequence on a chromosome.

The term “gene” refers to a DNA sequence that comprises control and coding sequences necessary for the production of an RNA having a non-coding function (e.g., a ribosomal or transfer RNA), a polypeptide, or a precursor. The RNA or polypeptide can be encoded by a full length coding sequence or by any portion of the coding sequence so long as the desired activity or function is retained. Thus, a “gene” refers to a DNA or RNA, or portion thereof, that encodes a polypeptide or an RNA chain that has functional role to play in an organism. For the purpose of this invention it may be considered that genes include regions that regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, and locus control regions.

The term “wild-type” refers to a gene or a gene product that has the characteristics of that gene or gene product when isolated from a naturally occurring source. A wild-type gene is that which is most frequently observed in a population and is thus arbitrarily designated the “normal” or “wild-type” form of the gene. In contrast, the term “modified,” “mutant,” or “polymorphic” refers to a gene or gene product that displays modifications in sequence and or functional properties (i.e., altered characteristics) when compared to the wild-type gene or gene product. It is noted that naturally-occurring mutants can be isolated; these are identified by the fact that they have altered characteristics when compared to the wild-type gene or gene product.

As used herein, the term “variant” should be taken to mean the exhibition of qualities that have a pattern that deviates from what occurs in nature.

The terms “non-naturally occurring” or “engineered” are used interchangeably and indicate the involvement of the hand of man. The terms, when referring to nucleic acid molecules or polypeptides mean that the nucleic acid molecule or the polypeptide is at least substantially free from at least one other component with which they are naturally associated in nature and as found in nature.

The term “oligonucleotide” as used herein is defined as a molecule comprising two or more deoxyribonucleotides or ribonucleotides, preferably at least 5 nucleotides, more preferably at least about 10 to 15 nucleotides and more preferably at least about 15 to 50 nucleotides (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more nucleotides). The exact size will depend on many factors, which in turn depend on the ultimate function or use of the oligonucleotide. The oligonucleotide may be generated in any manner, including chemical synthesis, DNA replication, reverse transcription, PCR, or a combination thereof.

Because mononucleotides are reacted to make oligonucleotides in a manner such that the 5′ phosphate of one mononucleotide pentose ring is attached to the 3′ oxygen of its neighbor in one direction via a phosphodiester linkage, an end of an oligonucleotide is referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. As used herein, a nucleic acid sequence, even if internal to a larger oligonucleotide, also may be said to have 5′ and 3′ ends. A first region along a nucleic acid strand is said to be upstream of another region if the 3′ end of the first region is before the 5′ end of the second region when moving along a strand of nucleic acid in a 5′ to 3′ direction.

When two different, non-overlapping oligonucleotides anneal to different regions of the same linear complementary nucleic acid sequence, and the 3′ end of one oligonucleotide points towards the 5′ end of the other, the former may be called the “upstream” oligonucleotide and the latter the “downstream” oligonucleotide. Similarly, when two overlapping oligonucleotides are hybridized to the same linear complementary nucleic acid sequence, with the first oligonucleotide positioned such that its 5′ end is upstream of the 5′ end of the second oligonucleotide, and the 3′ end of the first oligonucleotide is upstream of the 3′ end of the second oligonucleotide, the first oligonucleotide may be called the “upstream” oligonucleotide and the second oligonucleotide may be called the “downstream” oligonucleotide.

The terms “peptide” and “polypeptide” and “protein” are used interchangeably herein, and refer to a polymeric form of amino acids of any length, which can include coded and non-coded amino acids, chemically or biochemically modified or derivatized amino acids, and polypeptides having modified peptide backbones.

As used herein, the term “binding” (e.g., with reference to an RNA-binding domain of a polypeptide) refers to a non-covalent interaction between macromolecules (e.g., between a protein and a nucleic acid). While in a state of non-covalent interaction, the macromolecules are said to be “associated” or “interacting” or “binding” (e.g., when a molecule X is said to interact with a molecule Y, it is meant the molecule X binds to molecule Y in a non-covalent manner). Not all components of a binding interaction need be sequence-specific (e.g., contacts with phosphate residues in a DNA backbone), but some portions of a binding interaction may be sequence specific. Binding interactions are generally characterized by a dissociation constant (K_{d}) of less than 10⁻⁶ M, less than 10⁻⁷ M, less than 10⁻⁸ M, less than 10⁻⁹ M, less than 10⁻¹⁰ M, less than 10⁻¹¹ M, less than 10⁻¹² M, less than 10⁻¹³ M, less than 10⁻¹⁴ M, or less than 10⁻¹⁵ M. “Affinity” refers to the strength of binding, increased binding affinity being correlated with a lower K_{d}.

As used herein, the term “binding domain” refers to a protein domain that is able to bind non-covalently to another molecule. A binding domain can bind to, for example, a DNA molecule (a DNA-binding protein), an RNA molecule (an RNA-binding protein) and/or a protein molecule (a protein-binding protein). In the case of a protein domain-binding protein, it can bind to itself (to form homodimers, homotrimers, etc.) and/or it can bind to one or more molecules of a different protein or proteins.

As used herein, the term “ribonucleoprotein”, abbreviated “RNP” refers to a multimolecular complex comprising a polypeptide (e.g., a CRISPR protein such as, e.g., a Cas13 protein or a protein having an activity similar to a Cas13 protein) and a ribonucleic acid (e.g., a gRNA). In some embodiments, the polypeptide and ribonucleic acid are bound by a non-covalent interaction.

As used herein, the term “conservative amino acid substitution” refers to the interchangeability in proteins of amino acid residues having similar side chains. For example, a group of amino acids having aliphatic side chains consists of glycine, alanine, valine, leucine, and isoleucine; a group of amino acids having aliphatic-hydroxyl side chains consists of serine and threonine; a group of amino acids having amide containing side chains consisting of asparagine and glutamine; a group of amino acids having aromatic side chains consists of phenylalanine, tyrosine, and tryptophan; a group of amino acids having basic side chains consists of lysine, arginine, and histidine; a group of amino acids having acidic side chains consists of glutamate and aspartate; and a group of amino acids having sulfur containing side chains consists of cysteine and methionine. Exemplary conservative amino acid substitution groups are: valine-leucine/isoleucine, phenylalanine-tyrosine, lysine-arginine, alanine-valine, and asparagine-glutamine.

As used herein, the term “recombinant” refers to a particular nucleic acid (DNA or RNA) that is the product of various combinations of cloning, restriction, polymerase chain reaction (PCR) and/or ligation steps resulting in a construct having a structural coding or non-coding sequence distinguishable from endogenous nucleic acids found in natural systems. DNA sequences encoding polypeptides can be assembled from cDNA fragments or from a series of synthetic oligonucleotides, to provide a synthetic nucleic acid which is capable of being expressed from a recombinant transcriptional unit contained in a cell or in a cell-free transcription and translation system. Genomic DNA comprising the relevant sequences can also be used in the formation of a recombinant gene or transcriptional unit. Sequences of non-translated DNA may be present 5′ or 3′ from the open reading frame, where such sequences do not interfere with manipulation or expression of the coding regions, and may indeed act to modulate production of a desired product by various mechanisms). Alternatively, DNA sequences encoding RNA (e.g., DNA-targeting RNA) that is not translated may also be considered recombinant. Thus, e.g., the term “recombinant” nucleic acid refers to one which is not naturally occurring, e.g., is made by the artificial combination of two otherwise separated segments of sequence through human intervention. This artificial combination is often accomplished by either chemical synthesis means, or by the artificial manipulation of isolated segments of nucleic acids, e.g., by genetic engineering techniques. Such is usually done to replace a codon with a codon encoding the same amino acid, a conservative amino acid, or a non-conservative amino acid. Alternatively, it is performed to join together nucleic acid segments of desired functions to generate a desired combination of functions. This artificial combination is often accomplished by either chemical synthesis means, or by the artificial manipulation of isolated segments of nucleic acids, e.g., by genetic engineering techniques. When a recombinant polynucleotide encodes a polypeptide, the sequence of the encoded polypeptide can be naturally occurring (“wild type”) or can be a variant (e.g., a mutant) of the naturally occurring sequence. Thus, the term “recombinant” polypeptide does not necessarily refer to a polypeptide whose sequence does not naturally occur. Instead, a “recombinant” polypeptide is encoded by a recombinant DNA sequence, but the sequence of the polypeptide can be naturally occurring (“wild type”) or non-naturally occurring (e.g., a variant, a mutant, etc.). Thus, a “recombinant” polypeptide is the result of human intervention but may be a naturally occurring amino acid sequence.

As used herein, the term “vector” or “expression vector” refers to a replicon, such as a plasmid, phage, virus, or cosmid, to which another DNA segment (an “insert”) may be attached so as to bring about the replication of the segment in a cell.

A cell has been “genetically modified” or “transformed” or “transfected” by exogenous DNA, e.g., a recombinant expression vector, when such DNA has been introduced inside the cell. The presence of the exogenous DNA results in permanent or transient genetic change. The transforming DNA may or may not be integrated (covalently linked) into the genome of the cell. In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones that comprise a population of daughter cells containing the transforming DNA. A “clone” is a population of cells derived from a single cell or common ancestor by mitosis. A “cell line” is a clone of a primary cell that is capable of stable growth in vitro for many generations.

Suitable methods of genetic modification (also referred to as “transformation”) include e.g., viral or bacteriophage infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery (see, e.g., Panyam and Labhasetwar (2012), Advanced Drug Delivery Reviews, 64 (supplement): 61-71, incorporated herein by reference). The choice of method of genetic modification is generally dependent on the type of cell being transformed and the circumstances under which the transformation is taking place (e.g., in vitro, ex vivo, or in vivo). A general discussion of these methods can be found in Ausubel, et al., Short Protocols in Molecular Biology, 3rd ed., Wiley & Sons, 1995, incorporated herein by reference.

The term “target nucleic acid” (e.g., a “target RNA”) as used herein refers to a polynucleotide that comprises a “target site” or “target sequence.” The terms “target site” or “target sequence” are used interchangeably herein to refer to a nucleic acid sequence present in a target RNA to which a RNA-targeting segment of a RNA-targeting RNA will bind, provided sufficient conditions for binding exist. Suitable RNA/RNA binding conditions include physiological conditions normally present in a cell. Other suitable RNA/RNA binding conditions (e.g., conditions in a cell-free system) are known in the art; see, e.g., Sambrook, referenced herein and incorporated by reference.

The RNA molecule that binds to the polypeptide in the RNP and targets the polypeptide to a specific location within the target RNA is referred to herein as the “RNA targeting RNA” or “RNA-targeting RNA polynucleotide” (also referred to herein as a “guide RNA” or “gRNA”). A RNA-targeting RNA comprises two segments, a “RNA-targeting segment” and a “protein-binding segment.” In some embodiments, the gRNA comprises two RNAs (e.g., a dgRNA, e.g., a crRNA and a tracrRNA) and in some embodiments the gRNA comprises one RNA (e.g., a sgRNA).

A RNA-targeting RNA and a polypeptide form an RNP complex (e.g., bind via non-covalent interactions). The RNA-targeting RNA provides target specificity to the RNP complex by comprising a nucleotide sequence that is complementary to a sequence of a target RNA. The polypeptide of the RNP complex provides site-specific binding and, in some embodiments, labeling (e.g., for imaging). In other words, the polypeptide of the RNP is guided to a target RNA sequence by virtue of its association with the protein-binding segment of the RNA-targeting RNA.

In some embodiments, a RNA-targeting RNA comprises two separate RNA molecules (e.g., two RNA polynucleotides, e.g., an “activator-RNA” and a “targeter-RNA”) and is referred to herein as a “double-molecule RNA-targeting RNA” or a “two-molecule RNA-targeting RNA” or a “double guide RNA” or a “dgRNA”. In other embodiments, the RNA-targeting RNA is a single RNA molecule (e.g., a single RNA polynucleotide) and is referred to herein as a “single-molecule RNA-targeting RNA,” a “single guide RNA,” or an “sgRNA.” The term “RNA-targeting RNA” or “guide RNA” or “gRNA” is inclusive, referring both to double-molecule RNA-targeting RNAs (dgRNAs) and to single-molecule RNA-targeting RNAs (sgRNAs).

As used herein, “CRISPR system” refers collectively to transcripts and other elements involved in the expression of and/or directing the activity of CRISPR-associated (“Cas”) genes, including sequences encoding a Cas gene, a gRNA or other sequences and transcripts from a CRISPR locus. In embodiments of the invention, the terms guide sequence and guide RNA (gRNA) are used interchangeably.

As used herein, the terms “subject” and “patient” refer to any organisms including plants, microorganisms, and animals (e.g., mammals such as dogs, cats, livestock, and humans).

The terms “treatment”, “treating”, and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease or symptom in a mammal and includes: (a) preventing the disease or symptom from occurring in a subject which may be predisposed to acquiring the disease or symptom but has not yet been diagnosed as having it; (b) inhibiting the disease or symptom, e.g., arresting its development; or (c) relieving the disease, e.g., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy will desirably be administered during the symptomatic stage of the disease, and in some cases after the symptomatic stage of the disease

The term “sample” in the present specification and claims is used in its broadest sense. On the one hand it is meant to include a specimen or culture (e.g., microbiological cultures). On the other hand. it is meant to include both biological and environmental samples. A sample may include a specimen of synthetic origin.

As used herein, a “biological sample” refers to a sample of biological tissue or fluid. For instance, a biological sample may be a sample obtained from an animal (including a human); a fluid, solid, or tissue sample; as well as liquid and solid food and feed products and ingredients such as dairy items, vegetables, meat and meat by-products, and waste. Biological samples may be obtained from all of the various families of domestic animals, as well as feral or wild animals, including, but not limited to, such animals as ungulates, bear, fish, lagomorphs, rodents, etc. Examples of biological samples include sections of tissues, blood, blood fractions, plasma, serum, urine, or samples from other peripheral sources or cell cultures, cell colonies, single cells, or a collection of single cells. Furthermore, a biological sample includes pools or mixtures of the above mentioned samples. A biological sample may be provided by removing a sample of cells from a subject but can also be provided by using a previously isolated sample. For example, a tissue sample can be removed from a subject suspected of having a disease by conventional biopsy techniques. In some embodiments, a blood sample is taken from a subject. A biological sample from a patient means a sample from a subject suspected to be affected by a disease.

The term “label” as used herein refers to any atom or molecule that can be used to provide a detectable (preferably quantifiable) effect, and that can be attached to a nucleic acid or protein. Labels include, but are not limited to, dyes (e.g., fluorescent dyes or moieties); radiolabels such as ²P; binding moieties such as biotin; haptens such as digoxigenin; luminogenic, phosphorescent, or fluorogenic moieties; mass tags; and fluorescent dyes alone or in combination with moieties that can suppress or shift emission spectra by fluorescence resonance energy transfer (FRET). Labels may provide signals detectable by fluorescence, radioactivity, colorimetry, gravimetry, X-ray diffraction or absorption, magnetism, enzymatic activity, characteristics of mass or behavior affected by mass (e.g., MALDI time-of-flight mass spectrometry; fluorescence polarization), and the like. A label may be a charged moiety (positive or negative charge) or, alternatively, may be charge neutral. Labels can include or consist of nucleic acid or protein sequence, so long as the sequence comprising the label is detectable. In some embodiments, a label is a “contrast agent” used, e.g., for computerized tomography (CT), magnetic resonance imaging (MRI), ultrasound, X-ray based techniques, ultrasound, 20 optical imaging modalities, Overhauser MRI (OMRI), oxygen imaging (OXI), magnetic source imaging (MSI), applied potential tomography (APT), and imaging methods based on microwaves. Non-limiting examples of contrast agents include, e.g., radiocontrast agents (e.g., iodine, barium); gadolinium; 99m-technetium; magnetic materials; thallium; F-18 labeled molecules (e.g., ¹⁸F-labelled glucose ([¹⁸F]FDG)); and metal-chelate complexes.

As used herein, “moiety” refers to one of two or more parts into which something may be divided, such as, for example, the various parts of an oligonucleotide, a molecule, a chemical group, a domain, a probe, etc.

As used herein, a “stem-loop structure” refers to a nucleic acid having a secondary structure that includes a region of nucleotides that are known or predicted to form a double strand (stem portion) that is linked on one side to a region of predominantly single-stranded nucleotides (loop portion). The terms “hairpin” and “fold-back” structures are also used herein to refer to stem-loop structures. Such structures are well known in the art and these terms are used consistently with their known meanings in the art. As is known in the art, a stem-loop structure does not require exact base pairing. Thus, the stem may include one or more base mismatches. Alternatively, the base pairing may be exact, e.g., not include any mismatches As used herein, the term “modulate” means to produce a qualitative or quantitative change, e.g., in degree (e.g., to increase or decrease), in time (e.g., to cause to occur earlier or later), or in space (e.g., to change the location in one or more spatial dimension). In some embodiments, modulating refers to inhibiting or activating a biological molecule, pathway, or system. Accordingly, the term “modulate” can refer to an increase, a decrease, or other alteration of any, or all, chemical and/or biological activities or properties of a biochemical entity. As such, the term “modulate” can mean “inhibit”, “reduce”, or “suppress”, but the use of the word “modulate” is not limited to this definition. The term “modulation” as used herein refers to both upregulation (e.g., activation, enhancement, or stimulation) and downregulation (e.g., inhibition, reduction, or suppression), e.g., of a gene or genetic locus, e.g., by CRISPR/Cas13. Thus, the term “modulation”, when used in reference to a functional property or biological activity or process refers to the capacity to upregulate (e.g., activate, enhance, or stimulate), downregulate (e.g., inhibit, reduce, or suppress), or otherwise change a quality of such property, activity, or process.

As used herein, a “decrease” can refer to any change that results in a smaller amount of a symptom, composition, or activity. A substance is also understood to decrease the genetic output of a gene when the genetic output of the gene product with the substance is less relative to the output of the gene product without the substance.

Also, for example, a decrease can be a change in the symptoms of a disorder such that the symptoms are less than previously observed.

As used herein, an “increase” can refer to any change that results in a larger amount of a symptom, composition, or activity. A substance is also understood to increase the genetic output of a gene when the genetic output of the gene product with the substance is more relative to the output of the gene product without the substance.

Also, for example, an increase can be a change in the symptoms of a disorder such that the symptoms are more than previously observed. An increase can include but is not limited to a 10%, 20%, 30%, 40%, 50%, 60%, 70%, 80%, 90%, or 100% increase.

As used herein, the terms “inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

### DESCRIPTION

Nucleotide repeat elements are present in the genomes of eukaryotes including humans and expansions of these repeat elements can cause disease. For example, a C9orf72 hexanucleotide (e.g., GGGGCC) repeat expansion mutation is a common genetic cause of amyotrophic lateral sclerosis (ALS) and frontotemporal dementia (FTD) (see, e.g., Renton (2011) “A hexanucleotide repeat expansion in C90RF72 is the cause of chromosome 9p21-linked ALS-FTD” Neuron 72: 257-268; and DeJesus-Hernandez (2011) “Expanded GGGGCC hexanucleotide repeat in noncoding region of C90RF72 causes chromosome 9p-linked FTD and ALS” Neuron 72: 245-256, each of which is incorporated herein by reference). Genetic evidence indicates also that the C9orf72 hexanucleotide repeat expansion (HRE) contributes to Alzheimer's disease (see, e.g., Kohli (2012) “Repeat expansions in the C90RF72 gene contribute to Alzheimer's disease in Caucasians” Neurobiology of Aging 34(5)1519.e5-12; Majounie (2012) “Repeat expansion in C90RF72 in Alzheimer's disease” N Engl J Med 366: 283-284; Rollinson (2012) “Analysis of the hexanucleotide repeat in C90RF72 in Alzheimer's disease” Neurobiology of Aging 33: 1846 e1845-1846; and Harms (2013) “C9orf72 hexanucleotide repeat expansions in clinical Alzheimer disease” JAMA neurology 70: 736-741, each of which is incorporated herein by reference), Huntington's disease (see, e.g., Hensman Moss (2014) “C9orf72 expansions are the most common genetic cause of Huntington disease phenocopies” Neurology 82: 292-299, incorporated herein by reference), and other neurological conditions including multiple system atrophy, depressive pseudo dementia, and bipolar disorder (see, e.g., Goldman (2014) “Multiple System Atrophy and Amyotrophic Lateral Sclerosis in a Family With Hexanucleotide Repeat Expansions in C9orf72” JAMA neurology 71(6):771-4; Bieniek (2014) “Expanded C90RF72 Hexanucleotide Repeat in Depressive Pseudodementia” JAMA neurology 71(6):775-81; and Galimberti (2014) “C90RF72 hexanucleotide repeat expansion as a rare cause of bipolar disorder” Bipolar disorders 16: 448-449, each of which is incorporated herein by reference).

Hexanucleotide expansion is proposed to cause disease by three mechanisms: haploinsufficiency of C9orf72 protein (a loss-of-function mechanism); formation of RNA foci (a gain-of-function mechanism); and expression of dipeptide repeat proteins (DPRs) through repeat-associated non-AUG (RAN) translation (a gain-of-function mechanism) (see, e.g., Balendra (2018) “C9orf72-mediated ALS and FTD: multiple pathways to disease” Nat Rev Neurol 14(9):544-558, incorporated herein by reference). Accordingly, therapeutic strategies including antisense oligonucleotides (ASOs), RNA-targeting Cas9 (RCas9), siRNAs, artificial microRNAs, and DPR antibodies have been developed by targeting either repeat associated RNAs or DPRs (see, e.g., Jiang (2016) “Gain of Toxicity from ALS/FTD-Linked Repeat Expansions in C90RF72 Is Alleviated by Antisense Oligonucleotides Targeting GGGGCC-Containing RNAs” Neuron 90: 535-550; Batra (2017) “Elimination of Toxic Microsatellite Repeat Expansion RNA by RNA-Targeting Cas9” Cell 170: 899-912 e810; Martier (2019) “Targeting RNA-Mediated Toxicity in C9orf72 ALS and/or FTD by RNAi-Based Gene Therapy” Mol Ther Nucleic Acids 16: 26-37; Martier (2019) “Artificial MicroRNAs Targeting C9orf72 Can Reduce Accumulation of Intranuclear Transcripts in ALS and FTD Patients” Mol Ther Nucleic Acids 14: 593-608; and Nguyen (2020) “Antibody Therapy Targeting RAN Proteins Rescues C9 ALS/FTD Phenotypes in C9orf72 Mouse Model” Neuron 105: 645-662 e611, each of which is incorporated herein by reference).

Although these strategies for managing expression of aberrant C9orf72 transcripts have shown some promising results, limitations still exist. For example, ASOs must be routinely administered for the life of the patient; RCas9 has a low efficiency for GGGGCC repeat RNA; siRNAs and miRNAs do not directly target repeat RNA; and therapeutic antibodies are expensive. Further, while therapies using CRISPR systems have shown promise for knock-down of gene expression with low off-target activity in several therapeutic applications (see, e.g., Konermann (2018) “Transcriptome Engineering with RNA-Targeting Type VI-D CRISPR Effectors” Cell 173: 665-676 e614, incorporated herein by reference), some specific CRISPR targets have been particularly difficult to target for knock-down. For instance, CRISPR technologies have not been successfully used for targeting aberrant C9orf72 transcripts that contain hexanucleotide repeats of GGGGCC and that have complicated higher-order structures (e.g., G-quadruplexes and hairpins) (see, e.g., Haeusler (2014) “C9orf72 nucleotide repeat structures initiate molecular cascades of disease” Nature 507: 195-200, incorporated herein by reference).

The technology provided herein relates to a CRISPR-Cas13 system that targets hexanucleotide repeat RNAs, e.g., such as aberrant C9orf72 RNAs that contain GGGGCC repeats. Thus, in some embodiments, the CRISPR-Cas13 system described herein provides a technology for decreasing, minimizing, and/or eliminating toxic transcripts and/or DPR in patient cells. In some embodiments, the technology described herein provides a therapy for neurological diseases (e.g., ALS, FTD, Alzheimer's disease, Huntington's disease, multiple system atrophy, depressive pseudo dementia, and bipolar disorder) caused by hexanucleotide (e.g., GGGGCC) expansion at particular genetic loci (e.g., C9orf72).

Embodiments of the technology described herein provide a new method of using CRISPR (e.g., CRISPR-Cas13) to decrease the amount of toxic RNA and protein products generated from GGGGCC repeats associated with various neurological diseases including ALS and FTD. The technology comprises use of CasRx sequences that were optimized during the development of the technology for targeting the GGGGCC repeats. The technology finds use in treating C9orf72-linked ALS/FTD. Furthermore, the technology finds use in treating other repeat expansion diseases.

### CRISPR-Cas13

CRISPR-Cas13 is a Type VI CRISPR-Cas protein that targets RNA using a single CRISPR RNA (crRNA) (see, e.g., Abudayyeh cited herein). To date, four subtypes of Cas13 proteins have been identified and been applied for gene knock-down, RNA imaging and tracking, viral RNA detection, site directed RNA editing (see, e.g., Cox cited herein), and RNA splicing alteration. Cas13d is the smallest Cas13 variant known to date and has been shown to exert robust gene knock-down efficiency with greatly reduced off-target activity compared to RNA interference. See, e.g., Abudayyeh (2016) “C2c2 is a single-component programmable RNA-guided RNA-targeting CRISPR effector” Science 353(6299):aaf5573; Konermann (2018) “Transcriptome Engineering with RNA-Targeting Type VI-D CRISPR Effectors” Cell 173: 665-676 e614; Yang (2019) “Dynamic Imaging of RNA in Living Cells by CRISPR-Cas13 Systems” Mol Cell 76: 981-997 e987; Freije (2019) “Programmable Inhibition and Detection of RNA Viruses Using Cas13” Mol Cell 76: 826-837 e811; and Cox (2017) “RNA editing with CRISPR-Cas13” Science 358: 1019-1027,each of which is incorporated herein by reference).

In some embodiments, the technology comprises use of an RNA-targeting protein (e.g., Cas13 (e.g., a Cas13a, Cas13b, Cas13c, Cas13d, CasRx, etc.)), which works according to a similar mechanism as Cas9. In addition to targeting genomic DNA, Cas9 and other CRISPR related proteins (e.g. Cas13) also target RNAs directed by gRNAs (see, e.g., Abudayyeh et al. (2017) “RNA targeting with CRISPR-Cas13” Nature 550: 280; Konermann (2018) “Transcriptome Engineering with RNA-Targeting Type VT-D CRISPR Effectors” Cell 173: 665-76, Yan (2018) “Cas13d Is a Compact RNA-Targeting Type VI CRISPR Effector Positively Modulated by a WYL-Domain-Containing Accessory Protein” Mol. Cell 70: 327-39, each of which is incorporated herein by reference). Thus, in some embodiments, labeled gRNAs complex with Cas13 or other RNA-guided nucleases (e.g., a class 2 type VI RNA-guided RNA-targeting CRISPR-Cas effector (e.g., Cas13), a dCpf1, etc.) to visualize and track dynamics of sequence-specific RNA transcripts and non-coding RNAs in cells. Accordingly, in some embodiments, the technology relates to labeling RNAs using fluorescent guide RNAs in complex with a protein to form an RNP (e.g., a fRNP), e.g., comprising a Cas13 or an RNA-targeting Cas13 (e.g., a dCas13, etc.) In some embodiments, the technology provided herein comprises used of a “dead” Cas13 protein, e.g., a dCas13d. The dCas13/gRNA complex binds to a target nucleic acid with a sequence specificity provided by the gRNA, but does not cleave the nucleic acid. In this form, the dCas13/gRNA RNP binds to the target nucleic acid with sequence specificity; in some embodiments, the RNP “melts” the target sequence to provide single-stranded regions of the target nucleic acid in a sequence-specific manner.

In some embodiments, the Cas13 is PspCas13b, PspCas13b Truncation, AdmCas13d, AspCas13b, AspCas13c, BmaCas13a, BzoCas13b, CamCas13a, CcaCas13b, Cga2Cas13a, CgaCas13a, EbaCas13a, EreCas13a, EsCas13d, FbrCas13b, FnbCas13c, FndCas13c, FnfCas13c, FnsCas13c, FpeCas13c, FulCas13c, HheCas13a, LbfCas13a, LbmCas13a, LbnCas13a, LbuCas13a, LseCas13a, LshCas13a, LspCas13a, Lwa2casl3a, LwaCas13a, LweCas13a, PauCas13b, PbuCas13b, PgiCas13b, PguCas13b, Pin2Cas13b, Pin3Cas13b, PinCas13b, Pprcasl3a, PsaCas13b, PsmCas13b, RaCas13d, RanCas13b, RcdCas13a, RcrCas13a, RcsCas13a, RfxCas13d, UrCas13d, dPspCas13b, PspCas13b_A133H, PspCas13b_A058H, dPspCas13b truncation, dAdmCas13d, dAspCas13b, dAspCas13c, dBmaCas13a, dBzoCas13b, dCamCas13a, dCcaCas13b, dCga2Cas13a, dCgaCas13a, dEbaCas13a, dEreCas13a, dEsCas13d, dFbrCas13b, dFnbCas13c, dFndCas13c, dFnfCas13c, dFnsCas13c, dFpeCas13c, dFulCas13c, dHheCas13a, dLbfCas13a, dLbmCas13a, dLbnCas13a, dLbuCas13a, dLseCas13a, dLshCas13a, dLspCas13a, dLwa2cas13a, dLwaCas13a, dLweCas13a, dPauCas13b, dPbuCas13b, dPgiCas13b, dPguCas13b, dPin2Cas13b, dPin3Cas13b, dPinCas13b, dPprCas13a, dPsaCas13b, dPsmCas13b, dRaCas13d, dRanCas13b, dRcdCas13a, dRcrCas13a, dRcsCas13a, dRfxCas13d, or dUrCas13d. Additional Cas proteins are known in the art (see, e.g., Konermann (2018) Cell 173: 665-676 e14; Yan (2018) Mol Cell 7:327-339 e5; Cox (2017) Science 358: 1019-1027; Abudayyeh (2017) Nature 550: 280-284; Gootenberg (2017) Science 356: 438-442; and East-Seletsky (2017) Mol Cell 66: 373-383 e3, each of which is incorporated herein by reference).

In some embodiments, the Cas13 is codon-optimized Cas13 for expression in mammalian and human cells.

### Targets

The technology described herein targets RNA molecules. In some embodiments, the Cas13/gRNA targets an RNA transcript (e.g., an mRNA, a non-coding RNA (e.g., rRNA, microRNA, tRNA, siRNA, snoRNA, exRNA, scaRNA, piRNA, shRNA, Xist, HOTAIR, short non-coding RNA, long non-coding RNA, etc.))

### Cells and Organisms

The technology is not limited in the biological system in which the technology finds use. In some embodiments, the technology finds use in a variety of cells. In some embodiments, the technology finds use in a prokaryotic cell; in some embodiments, the technology finds use in a eukaryotic cell. In some embodiments, the eukaryotic cell is a human cell. In some embodiments, the human cell is a patient cell.

In some embodiments, the technology finds use in a bacterial and/or an archaeal cell. In some embodiments, the technology finds use in a cell from a fungus, e.g., a yeast (e.g., S. cerevisiae, S. pombe), a fruit fly (e.g., D. melanogaster), a nematode (e.g., C. elegans), or a mammal such as a mouse, rat, monkey, or human. In some embodiments, the technology finds use in a cell from a plant, e.g., A. thaliana. In some embodiments, the technology finds use in a zebrafish.

In some embodiments, the technology finds use in a cultured cell, e.g., HeLa cells, CHO cells, HEK293 cells, 3T3 cells, stem cells (human embryonic stem cells, induced pluripotent stem cells), primary cells (fibroblasts, epithelial cells), etc.

In some embodiments, the technology finds use in a cell, tissue, etc. from an organism that has a genome sequence that is known.

In some embodiments, the technology relates to modifying an organism or mammal including human or a non-human mammal or organism by manipulation of a target sequence in a genomic locus of interest. In some embodiments, the modifications (e.g., perturbations) are applied to the organism as a whole or a single cell or population of cells from that organism (if the organism is multicellular). In some embodiments, a single cell or a population of cells is modified ex vivo and re-introduced to the organism. In some embodiments, the technology comprises in vivo embodiments.

### Delivery

In some embodiments, the technology comprises delivering one or more polynucleotides, such as a vector, a transcript, and/or a protein, to a host cell. In some embodiments, the technology further provides cells produced by such methods, and animals comprising or produced from such cells. In some embodiments, a CRISPR enzyme in combination with (and optionally complexed with) a guide sequence is delivered to a cell. Embodiments comprise use of viral and non-viral based gene transfer methods to introduce nucleic acids into cells or target tissues. In some embodiments, methods are used to administer nucleic acids encoding components of a CRISPR system to cells in culture, or in a host organism. Embodiments comprise use of non-viral vector delivery systems including DNA plasmids, RNA (e.g., a transcript of a vector described herein), naked nucleic acid, and nucleic acid complexed with a delivery vehicle, such as a liposome.

Some embodiments comprise use of a viral delivery system (e.g., a DNA or RNA virus; (lentivirus, retrovirus, adenovirus, e.g., adeno-associated virus), which have either episomal or integrated genomes after delivery to the cell. For a review of gene therapy procedures, see Anderson, Science 256:808-813 (1992); Nabel & Feigner, TIBTECH 1 1:21 1-217 (1993); Mitani & Caskey, TIBTECH 1 1: 162-166 (1993); Dillon, TIBTECH 1 1: 167-175 (1993); Miller, Nature 357:455-460 (1992); Van Brunt, Biotechnology 6(30): 1149-1154 (1988); Vigne, Restorative Neurology and Neuroscience 8:35-36 (1995); Kremer & Perricaudet, British Medical Bulletin 51(1 5:31-44 (1995); Haddada et al., in Current Topics in Microbiology and Immunology Doerfler and Bohm (eds) (1995); and Yu et al., Gene Therapy 1: 13-26 (1994).

In some embodiments, the technology comprises use of a method of non-viral delivery of nucleic acids, e.g., lipofection, microinjection, biolistics, virosomes, liposomes, immunoliposomes, polycation or lipid/nucleic acid conjugates, naked DNA, artificial virions, transposons, transfection (lipid-mediated (cationic lipid-mediated), cationic polymers, calcium phosphate), plasmid, transient membrane poration (e.g., electroporation, nucleofection), integrated expression from a chromosome, gesicles, and agent-enhanced uptake of DNA. Lipofection is described in, e.g., U.S. Pat. Nos. 5,049,386, 4,946,787; and 4,897,355) and lipofection reagents are sold commercially (e.g., TRANSFECTAM and LIPOLECTIN). Cationic and neutral lipids that are suitable for efficient receptor-recognition lipofection of polynucleotides include those of Feigner, WO 91/17424; WO 91/16024, Delivery can be to cells (e.g. in vitro or ex vivo administration) or target tissues (e.g. in vivo administration). Gesicles are produced via co-overexpression of three components in a mammalian packaging cell: a nanovesicle-inducing glycoprotein, Cas9 endonuclease, and the sgRNA specific to the target gene. See Quinn (2016) “Gesicle Mediated Delivery of a Cas9-sgRNA Protein Complex” Molecular Therapy 24: 5126

In some embodiments, the RNP is delivered into cells using a technique or composition related to nucleofection, cell penetrating peptide, viral vesicles, cell surface tunneling protein, ultrasound, electroporation, cell squeezing, nanoparticles, gold or other metal particles, lipid particles, liposomes, viral transduction, viral particles, cell-cell fusion, ballistics, microinjection, and exosome intake.

In some embodiments, the Cas13 protein comprises a nuclear localization signal (NLS), e.g., an SV40 NLS, to direct the RNP to enter a nucleus. In some embodiments, the protein comprises an importin beta binding (IBB) domain sequence, e.g., to promote import of the polypeptide into a cell nucleus, e.g., by an importin (see, e.g., Lott and Cingolani (2011), Biochim Biophys Acta 1813(9): 1578-92, incorporated herein by reference). In some embodiments, the protein comprises at least one nuclear localization signal, e.g., an NLS comprising one or more basic amino acids, e.g., as known in the art (see, e.g., Lange et al., J. Biol. Chem., 2007, 282:5101-5105). For example, in some embodiments, the NLS is a monopartite NSL, e.g., PKKKRKV (SEQ ID NO: 2) or PKKKRRV (SEQ ID NO: 3). In another embodiment, the NLS is a bipartite sequence. In another embodiment, the NLS is KRPAATKKAGQAKKKK (SEQ ID NO: 4). Embodiments provide that the NLS is located at the N-terminus, the C-terminus, or in an internal location of the RNA-guided endonuclease.

In some embodiments, the NLS is a retrotransposon NLS. In some embodiments, the NLS is derived from Ty1, yeast GAL4, SKI3, L29 or histone H2B proteins, polyoma virus large T protein, VP1 or VP2 capsid protein, SV40 VP1 or VP2 capsid protein, Adenovirus E1 a or DBP protein, influenza virus NS1 protein, hepatitis virus core antigen or the mammalian lamin, c-myc, max, c-myb, p53, c-erbA, jun, Tax, steroid receptor or Mx proteins, Nucleoplasmin (NPM2), Nucleophosmin (NPM1), or simian virus 40 (“SV40”) T-antigen. In some embodiments, the NLS is a Ty1 or Ty1-derived NLS, a Ty2 or Ty2- derived NLS or a MAK11 or MAK11-derived NLS.

In some embodiments, the NLS is a Ty1-like NLS. For example, in some embodiments, the Ty1-like NLS comprises KKRX motif. In some embodiments, the Ty1-like NLS comprises KKRX motif at the N-terminal end. In some embodiments, the Ty1-like NLS comprises KKR motif. In some embodiments, the Ty1-like NLS comprises KKR motif at the C-terminal end. In some embodiments, the Ty1-like NLS comprises a KKRX and a KKR motif. In some embodiments, the Ty1-like NLS comprises a KKRX at the N-terminal end and a KKR motif at the C-terminal end. In some embodiments, the Ty1-like NLS comprises at least 20 amino acids. In some embodiments, the Ty1- like NLS comprises between 20 and 40 amino acids. In some embodiments, the NLS comprises two copies of the same NLS. For example, in some embodiments, the NLS comprises a multimer of a first Ty1-derived NLS and a second Ty1-derived NLS.

In some embodiments, the protein comprises a Nuclear Export Signal (NES). In some embodiments, the NES is attached to the N-terminal end of the Cas protein. In some embodiments, the NES localizes the protein to the cytoplasm for targeting cytoplasmic RNA. In some embodiments, the protein comprises a localization signal that localizes the protein to an organelle. In some embodiments, the localization signal localizes the protein to the nucleolus, ribosome, vesicle, rough endoplasmic reticulum, Golgi apparatus, cytoskeleton, smooth endoplasmic reticulum, mitochondria, vacuole, cytosol, lysosome, or centriole. A number of localization signals are known in the art.

In some embodiments, the protein comprises a localization signal that localizes the protein to an organelle or extracellularly. In some embodiments, the localization signal localizes the protein to the nucleolus, ribosome, vesicle, rough endoplasmic reticulum, Golgi apparatus, cytoskeleton, smooth endoplasmic reticulum, mitochondria, vacuole, cytosol, lysosome, or centriole. A number of localization signals are known in the art. Exemplary localization signals include, but are not limited to lx mitochondrial targeting sequence, 4x mitochondrial targeting sequence, secretory signal sequence (IL-2), myristylation, Calsequestrin leader, KDEL retention and peroxisome targeting sequence.

In some embodiments, the protein may contain a purification and/or detection tag. In some embodiments, the tag is on the N-terminal end of the protein. In some embodiments, the tag is a 3×FLAG tag.

In some embodiments, the Cas13 protein further comprises at least one cell-penetrating domain. In some embodiments, the cell-penetrating domain is a cell-penetrating peptide sequence derived from the HIV-1 TAT protein, e.g., GRKKRRQRRRPPQPKKKRKV (SEQ ID NO: 12). In some embodiments, the cell-penetrating domain is the TLM domain comprising the sequence PLSSIFSRIGDPPKKKRKV (SEQ ID NO: 13), which is a cell-penetrating peptide sequence derived from the human hepatitis B virus. In some embodiments, the cell-penetrating domain is an MPG domain comprising the sequence GALFLGWLGAAGSTMGAPKKKRKV (SEQ ID NO: 14) or GALFLGFLGAAGSTMGAWSQPKKKRKV (SEQ ID NO: 15). In an additional embodiment, the cell-penetrating domain is the Pep-1 domain comprising the sequence KETWWETWWTEWSQPKKKRKV (SEQ ID NO: 16). In some embodiments the cell-penetrating domain is VP22, a cell penetrating peptide from Herpes simplex virus, or a polyarginine peptide sequence. Embodiments provide that the cell-penetrating domain is located at the N-terminus, the C-terminus, or in an internal location of the protein.

In still other embodiments, the Cas13 comprises at least one marker domain. Non-limiting examples of marker domains include fluorescent proteins, purification tags, and epitope tags. In some embodiments, the marker domain is a fluorescent protein. Non-limiting examples of suitable fluorescent proteins include green fluorescent proteins (e.g., GFP, GFP-2, tagGFP, turboGFP, EGFP, Emerald, Azami Green, Monomeric Azami Green. CopGFP, AceGFP, ZsGreenl), yellow fluorescent proteins (e.g., YFP, EYFP, Citrine, Venus, YPet, PhiYFP, ZsYellowl), blue fluorescent proteins (e.g., EBFP, EBFP2, Azurite, mKalamal, GFPuv, Sapphire, T-sapphire), cyan fluorescent proteins (e.g. ECFP, Cerulean, CyPet, AmCyanl, Midoriishi-Cyan), red fluorescent proteins (mKate, mKate2, mPlum, DsRed monomer, mCherry, mRFP1, DsRed-Express, DsRed2, DsRed-Monomer, HcRed-Tandem, HcRedl, AsRed2, eqFP611, mRasberry, mStrawberry, Jred), and orange fluorescent proteins (mOrange, mKO, Kusabira-Orange, Monomeric Kusabira-Orange, mTangerine, tdTomato), or any other suitable fluorescent protein. In some embodiments, the marker domain is a purification tag and/or an epitope tag.

Exemplary tags include, but are not limited to, glutathione-S-transferase (GST), chitin binding protein (CBP), maltose binding protein, thioredoxin (TRX), poly(NANP), tandem affinity purification (TAP) tag, myc, AcV5, AU1, AUS, E, ECS, E2, FLAG, HA, nus, Softag 1, Softag 3, Strep, SBP, Glu-Glu, HSV, KT3, S, S1, T7, V5, VSV-G, 6×His, biotin carboxyl carrier protein (BCCP), and calmodulin.

In some embodiments, the protein is provided as a single polypeptide (e.g., a full Cas13 protein). In some embodiments, the protein is provided in multiple polypeptides, e.g., a split Cas13 protein provided in two parts, three parts, etc.

Although the disclosure herein refers to certain illustrated embodiments, it is to be understood that these embodiments are presented by way of example and not by way of limitation.

### EXAMPLES

Cas13 is a family of CRISPR proteins that specifically target RNA, including Cas13a, Cas13b, Cas13c, and Cas13d (see, e.g., Burmistrz (2020) “RNA-Targeting CRISPR-Cas Systems and Their Applications” Int J Mol Sci, 2020. 21(3), incorporated herein by reference). It was contemplated that Cas13 proteins could be used to target GGGGCC repeat RNAs in C9orf72-related ALS patients. In particular, experiments were conducted to test Cas13b and Cas13d proteins because these particular Cas13 proteins have been shown to have robust RNA cleavage abilities and minimal off-target activities in cells compared with other Cas13 proteins (see, e.g., Cox (2017) “RNA editing with CRISPR-Cas13” Science 358(6366): 1019-27; and Konermann (2018) “Transcriptome Engineering with RNA-Targeting Type VI-D CRISPR Effectors” Cell 173(3): 665-76 e14, each of which is incorporated herein by reference).

Previous studies have indicated that both Cas13b and Cas13d have sequence preference, thus suggesting that some RNAs might not be efficiently targeted by Cas13 proteins, especially RNAs comprising strong secondary structure (see, e.g., Smargon (2017) “Cas13b Is a Type VI-B CRISPR-Associated RNA-Guided RNase Differentially Regulated by Accessory Proteins Csx27 and Csx28” Mol Cell 65(4): 618-30 e7; and Wessels (2020) “Massively parallel Cas13 screens reveal principles for guide RNA design” Nat Biotechnol 38(6): 722-27, each of which is incorporated herein by reference).

For example, Cas13b RNA targeting is dependent on a double-sided PFS (protospacer flanking sequence) and RNA accessibility (see, e.g., Smargon supra), and Cas13d shows diminished target knockdown for “G”-dependent structures including G-quadruplex (see, e.g., Wessels supra). C9orf72 GGGGCC repeat RNAs are highly GC-rich RNAs that form stable G-quadruplex structures (see, e.g., Haeusler (2014) “C9orf72 nucleotide repeat structures initiate molecular cascades of disease” Nature 507(7491): 195-200; Liu (2021) “A Helicase Unwinds Hexanucleotide Repeat RNA G-Quadruplexes and Facilitates Repeat-Associated Non-AUG Translation” J Am Chem Soc 143(19): 7368-79; Reddy (2013) “The disease-associated r(GGGGCC)n repeat from the C9orf72 gene forms tract length-dependent uni- and multimolecular RNA G-quadruplex structures” J Biol Chem 288(14): 9860-06; and Fratta (2012) “C9orf72 hexanucleotide repeat associated with amyotrophic lateral sclerosis and frontotemporal dementia forms RNA G-quadruplexes” Sci Rep 2: 1016, each of which is incorporated herein by reference). These stable secondary structures make it highly challenging to target C9orf72 repeat RNA with Cas13b or Cas13d. However, it was contemplated that targeting C9orf72 repeat RNA might be successful through careful design and optimization of the guide RNA sequences. Accordingly, during the development of embodiments of the technology described herein, experiments were conducted in which 11 vectors were constructed (4 for Cas13b and 7 for Cas13d) and knockdown efficiency was tested in an EGFP reporter system in HEK293 cells.

### Methods

### EGFP Reporter Assay.

DNA constructs for enhanced green fluorescent protein (EGFP) reporter assays were produced using a pcDNA3.1 (V79020, ThermoFisher) backbone as previously described (NEED REFERENCE). For western blotting assays, protein samples from cells were resolved on 12% SDS-PAGE gels and then transferred to nitrocellulose membranes (Bio-Rad). The primary antibodies were an anti-GFP antibody (Invitrogen, GF28R) used at a dilution of 1:500 or an anti-B-actin antibody (Santa Cruz, sc-47778) used at a dilution of 1:1000; and the secondary antibodies was a donkey anti-mouse IgG (680 LT, 926-68022) 1:1000) antibody used at a 1:1000 dilution. Blots were imaged on an Odyssey system and the images were analyzed with Image Studio version 5.2 (LI·COR).

### Lentivirus Packaging and Infection.

A general lentiviral construct was produced by replacing the Cas9 coding sequence in plasmid lentiCRISPR v2 (Addgene plasmid #52961; see Sanjana (2014) “Improved vectors and genome-wide libraries for CRISPR screening” Nat Methods 11(8): 783-84, incorporated herein by reference) with a CasRx coding sequence from plasmid pXR001: EF1a-CasRx-2A-EGFP (Addgene plasmid #109049; see Konermann (2018) “Transcriptome Engineering with RNA-Targeting Type VI-D CRISPR Effectors” Cell 173(3):665-676.e14, incorporated herein by reference). Individual constructs expressing specific guide RNAs were constructed by inserting DNA sequences encoding an individual guide RNA into the general lentiviral construct. Nucleic acids encoding guide RNA sequences were produced by IDT. For lentivirus production, the lentiviral vector was cotransfected with the packing plasmid psPAX2 (Addgene plasmid #12260) and the envelope plasmid pMD2.G (Addgene plasmid #12259) into HEK293 cells at a molar ratio of 1:1:1. Cells were transferred to fresh medium 6 hours after the transfection, and the transfected cells were grown for 48 hours. Supernatants containing the virus were then collected and filtered through 0.45-μm membrane (Millipore Sigma HVHP02500). The filtered supernatants were mixed with 4× Lentivirus Concentrator Solution (1×PBS (pH 7.4) and 40% PEG-8000 (w/v)) and left at 4° C. overnight. The resulting solution was centrifuged at 1,600×g for 60 minutes at 4° C.; the virus pellets were resuspended in cold PBS using 1/20 of the original volume and then aliquoted for storage. HeLa Flp-In cells and iPSCs were transduced with an aliquot of the 20× concentrated lentiviruses 24 hours after cell seeding in culture plates. Puromycin (2 μg/mL) was added 48 hours after transduction, and cells were selected with the drug for 7 days before harvesting.

### Poly-GP Detection.

Poly-GP detection was performed as previously described (NEED REFERENCE). Cells were lysed in RIPA buffer (Sigma, R0278) containing 1× proteinase inhibitor (Roche, cOmplete, EDTA-free) and centrifuged at 16,000×g for 20 minutes at 4° C. The supernatants were collected, and protein concentrations were quantified with the Pierce BCA protein assay (Thermo Scientific). The samples were diluted to the concentration of I mg/mL for ELISA. Briefly, 0.375 μg/mL of biotinylated rabbit anti-GP antibody was incubated in 96-well small spot streptavidin coated plates for 1 hour at room temperature. Following three PBST washes, 35 μL of cell lysate was added per well in duplicate and incubated for 3 hours at room temperature. After three PBST washes, sulfo-tagged detection antibody was added at 1 μg/mL, and the mixture was incubated for 1 hour. Following three PBST washes, 150 μL of read buffer was added, and the samples were immediately imaged by MESO QuickPlex SQ 120. Specificity was verified using lysates of HEK293 cells overexpressing GFP-tagged dipeptide repeat proteins. All reagents were from Meso Scale Discovery (MSD).

### Guide RNA Design.

Guide RNAs comprised a CRISPR direct repeat (DR) sequence and a spacer sequence. The DR mediates binding with the Cas13 enzyme, and the spacer sequence is specific for the target RNA. During the development of the technology described herein, guide RNAs were designed and optimized through testing numerous guide RNAs having different spacer RNA lengths and different spacer RNA sequences.

Guide RNAs were designed to produce a hairpin structure in the DR (e.g., the DR sequence was designed to have a low free energy associated with hairpin formation) and to avoid secondary structures in the spacer RNA (e.g., the spacer RNA sequence was designed to have a high free energy associated with formation of secondary structure). Several computational modeling tools are available for predicting RNA structures from RNA sequences. See, e.g., Zuker (1999) “Algorithms and Thermodynamics for RNA Secondary Structure Prediction: A Practical Guide” in Barciszewski and Clark (eds) RNA Biochemistry and Biotechnology, NATO Science Series (Series 3: High Technology), vol 70. Springer, Dordrecht; Liu (2020) “Computational approaches for effective CRISPR guide RNA design and evaluation” Computational and Structural Biotechnology Journal 18: 35-44; and Riesenberg (2022) “Improved gRNA secondary structures allow editing of target sites resistant to CRISPR-Cas9 cleavage” Nat Commun 13: 489, each of which is incorporated herein by reference. In particular, the mfold package may be used to predict free energies associated with formation of secondary structures in RNA. See, e.g., Mathews (1999) “Expanded Sequence Dependence of Thermodynamic Parameters Improves Prediction of RNA Secondary Structure” J. Mol. Biol. 288: 911-40, incorporated herein by reference.

Guide RNAs were designed to minimize repeated C or G bases. For instance, a sequence of “CCGGCCCCGGCC” was preferred over a sequence of “CCCCGGCCCCGG”.

Further, guide RNAs incorporate a transcription start site (+1 position) for the U6 promoter comprising a G or A at the +1 position to provide high expression (see, e.g., Gao (2017) “Mutation of nucleotides around the +1 position of type 3 polymerase III promoters: The effect on transcriptional activity and start site usage” Transcription 8(5): 275-87, incorporated herein by reference). In particular, when spacer RNA is at the 5′ end of the DR sequence for Cas13b, the spacer RNA comprises the G or A at the +1 position.

During the development of embodiments of the technology described herein, several guide RNA sequences were designed and tested. The DNA nucleotide sequences encoding the guide RNAs are provided in Table 1.

TABLE 1
guide RNAs

| gRNA name | gRNA sequence (5′ to 3′) | SEQ ID: |
|---|---|---|
| Cas13b-NT20 | GGTGATAAGTGGAATGCCATGgttgtggaa | 1 |
| | ggtccagttttgaggggctattacaac | |
| Cas13b-S20 | GCCCCGGCCCCGGCCCCGGCCgttgtggaa | 2 |
| | ggtccagttttgaggggctattacaac | |
| Cas13b-S22 | CCCGGCCCCGGCCCCGGCCCCgttgtggaa | 3 |
| | ggtccagttttgaggggctattacaac | |
| Cas13b-S24 | GGCCCCGGCCCCGGCCCCGGCCCCgttgtg | 4 |
| | gaaggtccagttttgaggggctattacaac | |
| Cas13d-NT20 | Gaacccctaccaactggtcggggtttgaaa | 5 |
| | cGTGATAAGTGGAATGCCATG | |
| Cas13d-S20 | Gaacccctaccaactggtcggggtttgaaa | 6 |
| | cCCCCGGCCCCGGCCCCGGCC | |
| Cas13d-S22 | Gaacccctaccaactggtcggggtttgaaa | 7 |
| | cCCCCGGCCCCGGCCCCGGCCCC | |
| Cas13d-S24 | Gaacccctaccaactggtcggggtttgaaa | 8 |
| | cCCGGCCCCGGCCCCGGCCCCGGCC | |
| Cas13d-S30 | Gaacccctaccaactggtcggggtttgaaa | 9 |
| | cCCGGCCCCGGCCCCGGCCCCGGCCCCGGC | |
| | C | |
| Cas13d-NT30 | Gaacccctaccaactggtcggggtttgaaa | 10 |
| | cTCACCAGAAGCGTACCATACTCACGAACA | |
| | G | |

### Example 1

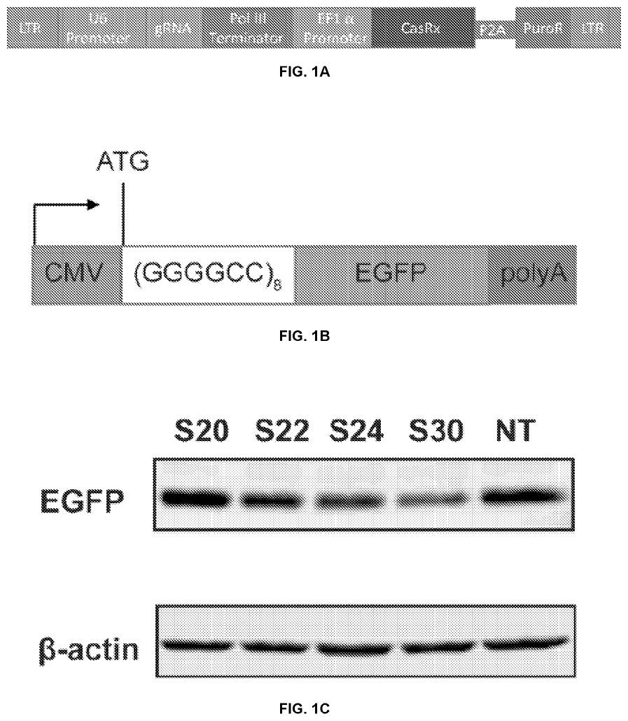

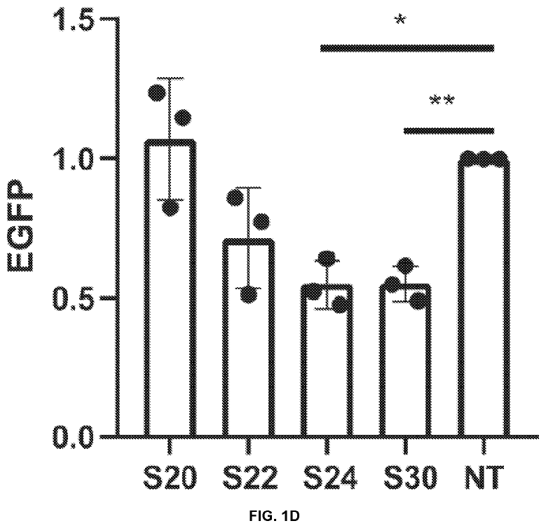

During the development of embodiments of the technology described herein, experiments were conducted to construct and test vectors comprising a sequence encoding a Cas13d protein and a sequence encoding a gRNA. See FIG. 1A. Since GGGGCC repeat RNA could adopt a highly thermodynamically stable G-quadruplex or hairpin structure (see, e.g., Reddy (2013) “The disease-associated r(GGGGCC)n repeat from the C9orf72 gene forms tract length-dependent uni- and multimolecular RNA G-quadruplex structures” J Biol Chem 288: 9860-9866; and Wang (2019) “The Hairpin Form of r(G4C2)(exp) in c9ALS/FTD Is Repeat-Associated Non-ATG Translated and a Target for Bioactive Small Molecules” Cell Chem Biol 26: 179-190 e112, each of which is incorporated herein by reference), and the secondary structure of gRNA might affect the guide RNA efficiency (see, e.g., Wessels (2020) “Massively parallel Cas13 screens reveal principles for guide RNA design.” Nature biotechnology 38: 722-727, incorporated herein by reference), the complementary spacer RNA was optimized by altering the length and sequence of the spacer RNA. To test the knock-down efficiency, a CRISPR-Cas13d construct (from a candidate pool) and a (GGGGCC)8-EGFP fusion expression construct were co-transfected into IIEK293 cells. The EGFP protein level was measured after 48 hours.

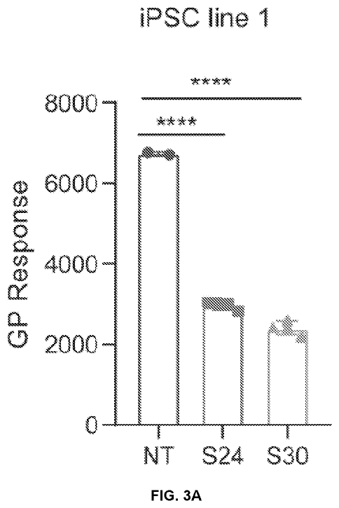

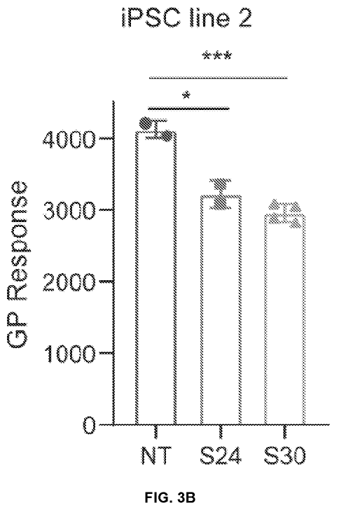

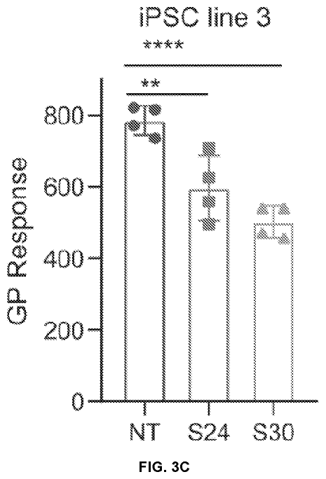

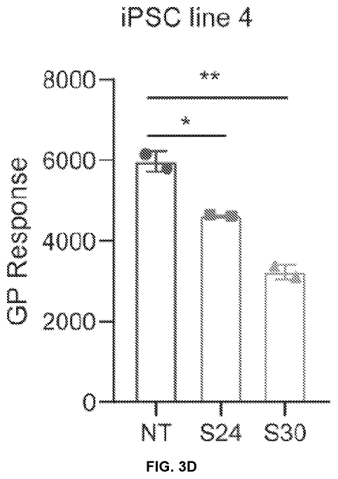

Data collected during the experiment indicated that the CRISPR-Cas13d construct worked as predicted. In particular, it was predicted that the CRISPR-Cas13d construct would produce a Cas13d/gRNA that would cleave the (GGGGCC)8 RNA and thus cause the EGFP mRNA not to be translated due to the absence of the start codon ATG. As shown by FIG. 1C, FIG. 1D, FIG. 213, and FIG. 3A-3E, the S24 and S30 gRNA significantly decreased EGFP expression and the S20 and S22 gRNA produced little or no change in EGFP expression compared to the gRNA with a non-targeting (NT) spacer control.

### Example 2

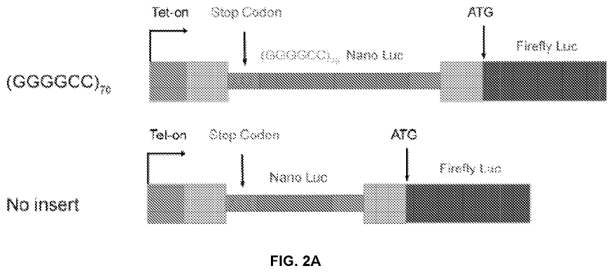

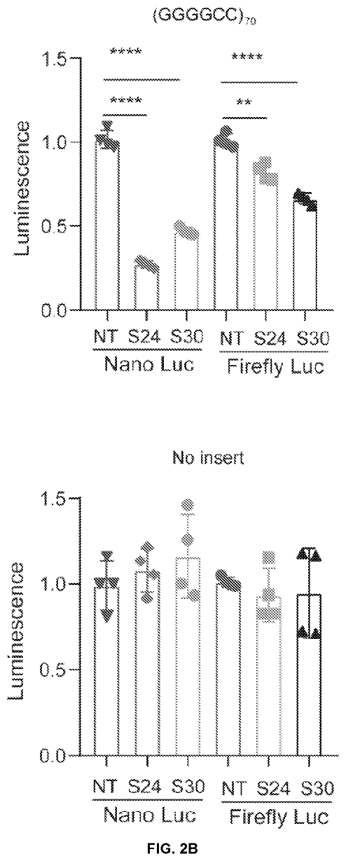

During the development of embodiments of the technology described herein, experiments were conducted to validate the CRISPR-Cas13d efficiency in vivo. In particular, data were collected from experiments using a dual-luciferase-based reporter cell line designed to monitor C9orf72 RAN translation. As shown in FIG. 2A, the reporter cell line comprised a test construct comprising a (GGGGCC)70 repeat DNA sequence placed before an ATG-lacking coding sequence of Nano luciferase (NLuc) and followed by an ATG-containing coding sequence of Firefly luciferase (FLuc) (see, e.g., Cheng (2018) “C90RF72 GGGGCC repeat-associated non-AUG translation is upregulated by stress through eTF2alpha phosphorylation” Nature communications 9: 51, incorporated herein by reference). A “No insert” control construct comprised the ATG-lacking coding sequence of Nano luciferase (NLuc) and the ATG-containing coding sequence of Firefly luciferase (FLuc) as in the test construct but lacked the (GGGGCC)70 repeat DNA sequence. (FIG. 2A). The “No insert” control construct was introduced into the reporter cell line to produce “No insert” control cells.

Lentivirus was used to introduce CRISPR-Cas13d constructs comprising test gRNA DNA sequences into the reporter cells and control cells to produce reporter cell lines stably expressing Cas13d and gRNAs. Data collected during the experiments indicated that both the S24 gRNA and S30 gRNA constructs significantly decreased the Nano luciferase signal in the reporter cell line comprising (GGGGCC)70 but the S24 gRNA and S30 gRNA constructs did not decrease the signal in the “No insert” control cell line (FIG. 2B). These data indicated that Cas13d guided by the S24 and S30 gRNA specifically recognized and cleaved the mRNA comprising the GGGGCC repeats. In addition, the data indicated that the firefly luciferase signal also decreased but decreased less than the decrease detected for the Nano luciferase. These data indicated that the Cas13d/gRNA degrades the spliced RNA comprising the (GGGGCC)70 and the data are consistent with the Cas13d/gRNA also degrading the pre-mRNA.

### Example 3

During the development of embodiments of the technology provided herein, experiments were conducted in vivo to test the CRISPR-Cas13d system to target repeat-containing RNA in cells derived from C9orf72-linked ALS patients. Four patient-derived human induced pluripotent stem cell (iPSC) lines and one B lymphocyte line were treated with lentivirus comprising sequences encoding Cas13d and gRNA. After puromycin selection for 7 days, cells were collected for GP detection with an ELISA method (see, e.g., Cheng (2019) “CRISPR-Cas9 Screens Identify the RNA Helicase DDX3X as a Repressor of C90RF72 (GGGGCC)n Repeat-Associated Non-AUG Translation” Neuron 104: 885-898 e888, incorporated herein by reference). The data indicated that the CRISPR-Cas13d system works in vivo in human patient cells with a GP knock-down efficiency from approximately 30% to 70%. FIG. 3A to 3D. Further, data indicated that the S30 gRNA provided a stronger knock down efficiency than the S24 gRNA.

### Example 4

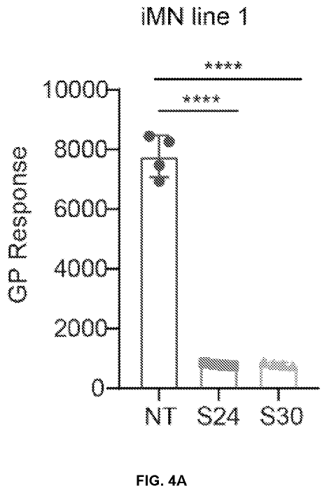

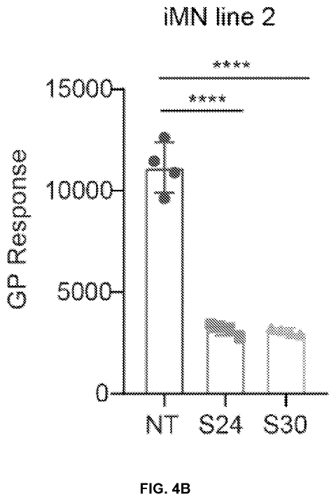

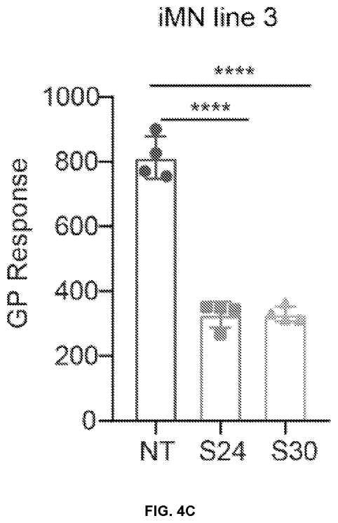

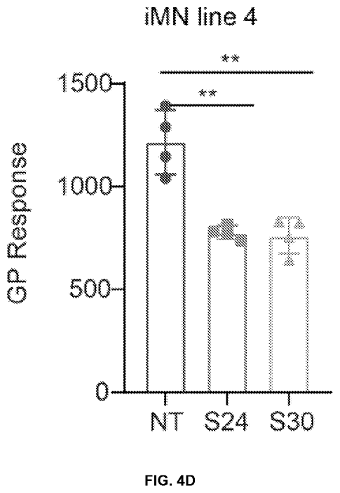

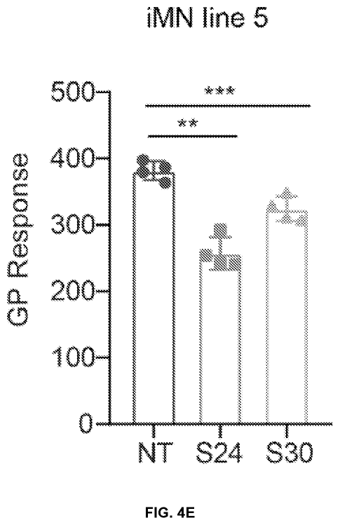

During the development of embodiments of the technology provided herein, experiments were conducted in vivo to test the CRISPR-Cas13d system to target repeat-containing RNA in motor neuron cells differentiated from iPSCs derived from C9orf72-linked ALS patients. Differentiation of iPSCs was performed according to Du, et al. (2015) “Generation and expansion of highly pure motor neuron progenitors from human pluripotent stem cells” Nat Commun 6: 6626, incorporated herein by reference. Five differentiated motor neuron cell lines were treated with lentivirus comprising sequences encoding Cas13d and gRNA for three days. After 11 days of culturing the cells, cells were harvested for GP analysis by an ELISA method (see, e.g., Cheng above). Consistent with the iPSC-based results described in Example 3, the data collected during these experiments indicated that the CRISPR-Cas13d system provided a GP knock-down efficiency of approximately 20% to 90% in human neurons in vivo. FIG. 4A to 4E.

All publications and patents mentioned in the above specification are herein incorporated by reference in their entirety for all purposes. Various modifications and variations of the described compositions, methods, and uses of the technology will be apparent to those skilled in the art without departing from the scope and spirit of the technology as described. Although the technology has been described in connection with specific exemplary embodiments, it should be understood that the invention as claimed should not be unduly limited to such specific embodiments. Indeed, various modifications of the described modes for carrying out the invention that are obvious to those skilled in the art are intended to be within the scope of the following claims.

We claim:

1. A ribonucleoprotein (RNP) comprising a Cas13 protein and a gRNA, wherein said gRNA comprises a spacer sequence comprising one or more CCGGCC sequences.

2. The RNP of claim 1, wherein said Cas13 protein is Cas13d.

3. The RNP of claim 1, wherein said spacer sequence is 24 nt long.

4. The RNP of claim 1, wherein said spacer sequence is 30 nt long.

5. The RNP of claim 1, wherein said gRNA sequence comprises a G or A at the +1 position.

6. The RNP of claim 1, wherein said gRNA comprises a sequence provided by SEQ TD NO: 8.

7. The RNP of claim 1, wherein said gRNA comprises a sequence provided by SEQ ID NO: 9.

8. A nucleic acid comprising a first nucleotide sequence encoding a Cas13 protein and a second nucleotide sequence encoding a gRNA, wherein said gRNA comprises a spacer sequence comprising one or more CCGGCC sequences.

9. The nucleic acid of claim 8, wherein said Cas13 protein is Cas13d.

10. The nucleic acid of claim 8, wherein said spacer sequence is 24 nt long.

11. The nucleic acid of claim 8, wherein said spacer sequence is 30 nt long.

12. The nucleic acid of claim 8, wherein said gRNA sequence comprises a G or A at the +1 position.

13. The nucleic acid of claim 8, wherein said gRNA comprises a sequence provided by SEQ ID NO: 8.

14. The nucleic acid of claim 8, wherein said gRNA comprises a sequence provided by SEQ ID NO: 9.

15. A vector comprising the nucleic acid of claim 8.

16. A cell comprising the nucleic acid of claim 8.

17. The cell of claim 16, wherein a vector comprises the nucleic acid.

18. The cell of claim 16, wherein the cell is a human cell comprising a hexanucleotide repeat in chromosome 9.

19. The cell of claim 16, wherein the cell is a human cell comprising a hexanucleotide repeat at a C9orf72 locus.

20. The cell of claim 16, wherein the cell is a human cell comprising one or more GGGGCC repeats at a C9orf72 locus.

21. The cell of claim 16, wherein a patient having a neurological disease comprises the cell.

22. The cell of claim 16, wherein a patient having amyotrophic lateral sclerosis comprises the cell.

23. The cell of claim 16, wherein a patient having frontotemporal dementia comprises the cell.

24. The cell of claim 16, wherein a patient having Alzheimer's disease comprises the cell.

25. The cell of claim 16, wherein a patient having Huntington's disease, multiple system atrophy, depressive pseudo dementia, or bipolar disorder comprises the cell.

26. A cell comprising the RNP of claim 1.

27. The cell of claim 26, wherein the cell is a human cell comprising a hexanucleotide repeat in chromosome 9.

28. The cell of claim 26, wherein the cell is a human cell comprising a hexanucleotide repeat at a C9orf72 locus.

29. The cell of claim 26, wherein the cell is a human cell comprising one or more GGGGCC repeats at a C9orf72 locus.

30. The cell of claim 26, wherein a patient having a neurological disease comprises the cell.

31. The cell of claim 26, wherein a patient having amyotrophic lateral sclerosis comprises the cell.

32. The cell of claim 26, wherein a patient having frontotemporal dementia comprises the cell.

33. The cell of claim 26, wherein a patient having Alzheimer's disease comprises the cell.

34. The cell of claim 26, wherein a patient having Huntington's disease, multiple system atrophy, depressive pseudo dementia, or bipolar disorder comprises the cell.

35. A system comprising a Cas13 protein and a gRNA, wherein said gRNA comprises a spacer sequence comprising one or more CCGGCC sequences.

36. The system of claim 35, wherein said Cas13 protein is Cas13d.

37. The system of claim 35, wherein said spacer sequence is 24 nt long.

38. The system of claim 35, wherein said spacer sequence is 30 nt long.

39. The system of claim 35, wherein said gRNA sequence comprises a G or A at the +1 position.

40. The system of claim 35, wherein said gRNA comprises a sequence provided by SEQ ID NO: 8.

41. The system of claim 35, wherein said gRNA comprises a sequence provided by SEQ ID NO: 9.

42. A system comprising a nucleic acid encoding a Cas13 protein and a gRNA, wherein said gRNA comprises a spacer sequence comprising one or more CCGGCC sequences.

43. The system of claim 42, wherein said Cas13 protein is Cas13d.

44. The system of claim 42, wherein said spacer sequence is 24 nt long.

45. The system of claim 42, wherein said spacer sequence is 30 nt long.

46. The system of claim 42, wherein said gRNA sequence comprises a G or A at the +1 position.

47. The system of claim 42 wherein said gRNA comprises a sequence provided by SEQ ID NO: 8.

48. The system of claim 42, wherein said gRNA comprises a sequence provided by SEQ ID NO: 9.

49. A system comprising a Cas13 protein and a nucleic acid encoding a gRNA, wherein said gRNA comprises a spacer sequence comprising one or more CCGGCC sequences.

50. The system of claim 49, wherein said Cas13 protein is Cas13d.

51. The system of claim 49, wherein said spacer sequence is 24 nt long.

52. The system of claim 49, wherein said spacer sequence is 30 nt long.

53. The system of claim 49, wherein said gRNA sequence comprises a G or A at the +1 position.

54. The system of claim 49, wherein said gRNA comprises a sequence provided by SEQ ID NO: 8.

55. The system of claim 49, wherein said gRNA comprises a sequence provided by SEQ ID NO: 9.

56. A system comprising a nucleic acid encoding a Cas13 protein and a nucleic acid encoding a gRNA, wherein said gRNA comprises a spacer sequence comprising one or more CCGGCC sequences.

57. The system of claim 56, wherein said Cas13 protein is Cas13d.

58. The system of claim 56, wherein said spacer sequence is 24 nt long.

59. The system of claim 56, wherein said spacer sequence is 30 nt long.

60. The system of claim 56, wherein said gRNA sequence comprises a G or A at the +1 position.

61. The system of claim 56, wherein said gRNA comprises a sequence provided by SEQ ID NO: 8.

62. The system of claim 56, wherein said gRNA comprises a sequence provided by SEQ ID NO: 9.

63. A method of treating a subject having a neurological disease, said method comprising administering a ribonucleoprotein (RNP) to said subject, wherein said RNP comprises a Cas13 protein and a gRNA and wherein said gRNA comprises a spacer sequence comprising one or more CCGGCC sequences.

64. The method of claim 63, wherein said Cas13 protein is Cas13d.

65. The method of claim 63, wherein said spacer sequence is 24 nt long.

66. The method of claim 63, wherein said spacer sequence is 30 nt long.

67. The method of claim 63, wherein said gRNA sequence comprises a G or A at the +1 position.

68. The method of claim 63, wherein said gRNA comprises a sequence provided by SEQ ID NO: 8.

69. The method of claim 63, wherein said gRNA comprises a sequence provided by SEQ ID NO: 9.

70. The method of claim 63, wherein said neurological disease is amyotrophic lateral sclerosis.

71. The method of claim 63, wherein said neurological disease is frontotemporal dementia.

72. The method of claim 63, wherein said neurological disease is Alzheimer's disease.

73. The method of claim 63, wherein said neurological disease is Huntington's disease, multiple system atrophy, depressive pseudo dementia, or bipolar disorder.

74. The method of claim 63, wherein said subject comprises a cell comprising a hexanucleotide repeat in chromosome 9.

75. The method of claim 63, wherein said subject comprises a cell comprising a hexanucleotide repeat at a C9orf72 locus.

76. The method of claim 63, wherein said subject comprises a cell comprising one or more GGGGCC repeats at a C9orf72 locus.

77. A method of treating a subject having a neurological disease, said method comprising administering a nucleic acid to said subject, wherein said nucleic acid comprises a first nucleotide sequence encoding a Cas13 protein and a second nucleotide sequence encoding a gRNA and wherein said gRNA comprises a spacer sequence comprising one or more CCGGCC sequences.

78. The method of claim 78, wherein said Cas13 protein is Cas13d.

79. The method of claim 78, wherein said spacer sequence is 24 nt long.

80. The method of claim 78, wherein said spacer sequence is 30 nt long.

81. The method of claim 78, wherein said gRNA sequence comprises a G or A at the +1 position.

82. The method of claim 78, wherein said gRNA comprises a sequence provided by SEQ ID NO: 8.

83. The method of claim 78, wherein said gRNA comprises a sequence provided by SEQ ID NO: 9.

84. The method of claim 78, wherein said neurological disease is amyotrophic lateral sclerosis.

85. The method of claim 78, wherein said neurological disease is frontotemporal dementia.

86. The method of claim 78, wherein said neurological disease is Alzheimer's disease.

87. The method of claim 78, wherein said neurological disease is Huntington's disease, multiple system atrophy, depressive pseudo dementia, or bipolar disorder.

88. The method of claim 78, wherein said subject comprises a cell comprising a hexanucleotide repeat in chromosome 9.

89. The method of claim 78, wherein said subject comprises a cell comprising a hexanucleotide repeat at a C9orf72 locus.

90. The method of claim 78, wherein said subject comprises a cell comprising one or more GGGGCC repeats at a C9orf72 locus.
